SEQUENCE LISTING (30 sequences)

SEQ ID NO: 1 - DNA, 4438 bp, synthetic construct
  misc_feature 1..4438: note = tadCas9
  source 1..4438: mol_type = other DNA
atggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgat

SEQ ID NO: 2 - protein, 1378 aa, synthetic construct
  REGION 1..1378: note = protein tadCas9
MAPKKKRKVGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - DNA, 103 bp, synthetic construct
  misc_feature 1..103: note = sgRNA M1
  source 1..103: mol_type = other DNA
cgggtgccgcactctcaacggttttagagctagaaatagcaagttaaaataaggctagtccgttatcaacttgaaaaagtggcaccgagtcggtgcttttttt

SEQ ID NO: 4 - DNA, 103 bp, synthetic construct
  misc_feature 1..103: note = sgRNA M2
  source 1..103: mol_type = other DNA
acagtgcacatgcatgcagcgttttagagctagaaatagcaagttaaaataaggctagtccgttatcaacttgaaaaagtggcaccgagtcggtgcttttttt

SEQ ID NO: 5 - protein, 132 aa, synthetic construct
  REGION 1..132: note = VP128
SGSETPGTSESATPESDALDDFDLDMLGSDALDDFDLDMLGSDALDDFDLDMLGSDALDDFDLDMLSGSETPGTSESATPESDALDDFDLDMLGSDALDDFDLDMLGSDALDDFDLDMLGSDALDDFDLDML

SEQ ID NO: 6 - protein, 318 aa, synthetic construct
  REGION 1..318: note = TALVP128
SGSETPGTSESATPESDEDPFAGAADDFPAFNEEELAWLMELLPQGSDEDPFAGAADDFPAFNEEELAWLMELLPQGSDEDPFAGAADDFPAFNEEELAWLMELLPQGSDEDPFAGAADDFPAFNEEELAWLMELLPQGSDEDPFAGAADDFPAFNEEELAWLMELLPQGSDEDPFAGAADDFPAFNEEELAWLMELLPQSGSETPGTSESATPESDALDDFDLDMLGSDALDDFDLDMLGSDALDDFDLDMLGSDALDDFDLDMLGSDALDDFDLDMLGSDALDDFDLDMLGSDALDDFDLDMLGSDALDDFDLDML

SEQ ID NO: 7 - protein, 116 aa, synthetic construct
  REGION 1..116: note = ZIM3 KRAB
SGSETPGTSESATPESMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 8 - protein, 28 aa, synthetic construct
  REGION 1..28: note = SRDX
SGSETPGTSESATPESLDLDLELRLGFA

SEQ ID NO: 9 - protein, 172 aa, synthetic construct
  REGION 1..172: note = KRAB::SRDX
SGSETPGTSESATPESMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGSLDLDLELRLGFAGSLDLDLELRLGFAGSLDLDLELRLGFAGSLDLDLELRLGFA

SEQ ID NO: 10 - DNA, 428 bp, synthetic construct
  misc_feature 1..428: note = NP1 1A
  source 1..428: mol_type = other DNA
tttattattttcttatagtttggaccgatctcctcacgtcatagttttgccttggtacctgtaatatgtatttgtgcttgtatataaaaatggagggagtatagttttttcgggagaaatatagtgcacgcgcaaaaccgtctcaagatcggccagtaaaccctcccccttgctgcttcatcgcatttgatatgatatactcaacagctcctcccgtctaaaaagaaagttcatcacttctgcatttcacaaccgatgcattccatttgatataaccattccctttctatactgacacaagtttatggggctgttctttgcactgcaaccgcctccgcattatttaacacatctaccctcgatctacccgcgcgctaactcgaggtttataaaccaagcgaacttttcgcgtccctgaagcgcaaagg

SEQ ID NO: 11 - DNA, 442 bp, synthetic construct
  misc_feature 1..442: note = NP11B
  source 1..442: mol_type = other DNA
tctttcaaaaaaaaacctcctgatgtactcagtgtcgtggccggtgatttaaccgtgtccactgattaattggaatgtatggtgttccaggtcaaaaaagaatgtcaaatgtaattctttcggaaaaaatatagtgcacgcgcaaaaccgtctcaagattggccagtaaaccctctcgcttgctgctccgtcgcatttgatatactcaacagctcctcctgtctaaaaagaaagttcatcacttctgcatttcacgaccaatgcattgcatttgatataaccattccctttctatagtaacacatgtttatgggctcctcgcggctcttctttgcactgcaaccgcctccgcattatttaacacatctaccctcgatctacccgcgcgctaactccaggtttataagcgaagcgaacttttcgcgtccctgaagcgtaaagg

SEQ ID NO: 12 - DNA, 432 bp, synthetic construct
  misc_feature 1..432: note = NP1 1D
  source 1..432: mol_type = other DNA
gttcgccagattatctttaccgctttcttttagtatctaggtttttcttagtattttattatttttctcatagtttggaccgatctcctcacgtcagaatgtcaaatgtaatcctttcgaaaaaaatatagtgcacgcgcgaaaaccgtctcaagattggccagtaaaccctcctgcttactgctccatcgcatttgatatactccttctgtgtaaaaagaatgttcatcacttttgcatttcacaaccaatgcattgcatttgatataaccattccctttctatagtaacacaagtttatgggctcctcgcagctgttctttgcactgcaaccgcctccgcattatttaacagatctaccctcgatctacccgcgcgctaactcgaggtttataaaccaagcgaacttttcgcgtccctgaagcgcaaagg

SEQ ID NO: 13 - DNA, 20 bp, synthetic construct
  misc_feature 1..20: note = sgRNA for 1A/1B
  source 1..20: mol_type = other DNA
gcgcaaaaccgtctcaagat

SEQ ID NO: 14 - DNA, 20 bp, synthetic construct
  misc_feature 1..20: note = sgRNA for 1B/1D
  source 1..20: mol_type = other DNA
ttccctttctatagtaacac

SEQ ID NO: 15 - DNA, 20 bp, synthetic construct
  misc_feature 1..20: note = sgRNA for 1A/1B/1D
  source 1..20: mol_type = other DNA
gttagcgcgcgggtagatcg

SEQ ID NO: 16 - DNA, 802 bp, synthetic construct
  misc_feature 1..802: note = MS2 promoter
  source 1..802: mol_type = other DNA
actgtctcgcgatttgagcatagagtcaatcgattttcctggccaatggcgtcaaggggagagatttggtcaaatgggcggaagtcgcagacccatgtatatgtgcacgggtgggtgggtgccttagggcatttacaacgcaaggcgctaaggcgggcgccagggtcaggatcctagtcgtttggcttagttcccgtccaaatttgagaattgagctggcatcgatgccatataagtcgtcgggcgtcgggcgctaactcagttttctgtctattttatgtgtgtagcgctcatacgtggctctcagcgttggaagagggactcttagcccaggcgctaggaagaaaatactattttatttccagtcaagtgcctgattaggcgccctccattggagatgcccttatgtgcctctctacgccgcagcagccggaaactacggcgcaccagtactggacggctcgtttcttattctaaacacagatactagtgtgcacctcgttgagagtgcggcacccgactgcacagtgcacatgcatgcagctggctctttctcttgacttgacacgctctcgcttctcccgattcctgcccgcgccggcgtctccacccgacttgatcgacatcggcatcggcatcggcctcggcatcggcccctcgacgacgctcagtatataagcgatcgggctggtggagctgcttgcagtacccgcagtggacacacgcttagctttagctacgtaggcgcagcagccggaaactagctagcaggtcgagaaggccggccggaggtagggagatg

SEQ ID NO: 17 - DNA, 4843 bp, synthetic construct
  misc_feature 1..4843: note = tadCas9::VP128
  source 1..4843: mol_type = other DNA
atggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgatggcgccagcggctctgagactccaggcacctctgagtctgctaccccagagtctgacgcgctggacgatttcgacctggatatgctgggcagcgacgccctggatgacttcgatcttgacatgctcggctccgacgctctcgacgactttgacctcgacatgttgggctctgatgcccttgacgattttgatttggacatgctgagcggcagcgaaaccccaggcacttcagaaagcgctacgccagagagcgacgcgttggacgactttgatctcgatatgctggggtccgatgcgctcgatgacttcgacttggatatgttgggttctgacgctttggatgattttgaccttgacatgcttggtagcgacgcactcgatgattttgatctggatatgctctag

SEQ ID NO: 18 - DNA, 13764 bp, synthetic construct
  misc_feature 1..13764: note = pBL- Ubi-tadCas9::VP128 M2
  source 1..13764: mol_type = other DNA
ctagttgatgagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaataagcttggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgtccggtttataaactcgcttgctgcatcagacttgacagtgcacatgcatgcagcgttttagagctagaaatagcaagttaaaataaggctagtccgttatcaacttgaaaaagtggcaccgagtcggtgctttttttgcatgcaagctgatccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagatcttggcaggatatattgtggtgtaacgtatcacaagtttgtacaaaaaagcaggctccgcggccgcgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcctgcagggttccggcggtagtggaggtagtggtggcctccaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcaagcttgcatgcctgcaggtccccagattagcggcgcgccgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgcccgggatccgccatggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgatggcgccagcggctctgagactccaggcacctctgagtctgctaccccagagtctgacgcgctggacgatttcgacctggatatgctgggcagcgacgccctggatgacttcgatcttgacatgctcggctccgacgctctcgacgactttgacctcgacatgttgggctctgatgcccttgacgattttgatttggacatgctgagcggcagcgaaaccccaggcacttcagaaagcgctacgccagagagcgacgcgttggacgactttgatctcgatatgctggggtccgatgcgctcgatgacttcgacttggatatgttgggttctgacgctttggatgattttgaccttgacatgcttggtagcgacgcactcgatgattttgatctggatatgctctaggctca

SEQ ID NO: 19 - DNA, 13764 bp, synthetic construct
  misc_feature 1..13764: note = pBL- Ubi-tadCas9::VP128 M1
  source 1..13764: mol_type = other DNA
ctagttgatgagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaataagcttggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgtccggtttataaactcgcttgctgcatcagacttgcgggtgccgcactctcaacggttttagagctagaaatagcaagttaaaataaggctagtccgttatcaacttgaaaaagtggcaccgagtcggtgctttttttgcatgcaagctgatccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagatcttggcaggatatattgtggtgtaacgtatcacaagtttgtacaaaaaagcaggctccgcggccgcgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcctgcagggttccggcggtagtggaggtagtggtggcctccaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcaagcttgcatgcctgcaggtccccagattagcggcgcgccgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgcccgggatccgccatggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgatggcgccagcggctctgagactccaggcacctctgagtctgctaccccagagtctgacgcgctggacgatttcgacctggatatgctgggcagcgacgccctggatgacttcgatcttgacatgctcggctccgacgctctcgacgactttgacctcgacatgttgggctctgatgcccttgacgattttgatttggacatgctgagcggcagcgaaaccccaggcacttcagaaagcgctacgccagagagcgacgcgttggacgactttgatctcgatatgctggggtccgatgcgctcgatgacttcgacttggatatgttgggttctgacgctttggatgattttgaccttgacatgcttggtagcgacgcactcgatgattttgatctggatatgctctaggctca

SEQ ID NO: 20 - DNA, 12301 bp, synthetic construct
  misc_feature 1..12301: note = pBL- OsLPT6-tadCas9::VP128 M2
  source 1..12301: mol_type = other DNA
ctagttgatgagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaataagcttggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgtccggtttataaactcgcttgctgcatcagacttgacagtgcacatgcatgcagcgttttagagctagaaatagcaagttaaaataaggctagtccgttatcaacttgaaaaagtggcaccgagtcggtgctttttttgcatgcaagctgatccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagatcttggcaggatatattgtggtgtaacgtatcacaagtttgtacaaaaaagcaggctccgcggccgcgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcctgcagggttccggcggtagtggaggtagtggtggcctccaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcaagcttgcatgcctgcaggtccccagattagcggcgcgccgaacctcagccaaaaccgaagacagtaccgccgaaggaccttatttaaacggttttgttaagttgggggacccatcgtacccggttttgcgaccggggacgaaaatcggactaggtgataaatagagggacccaaagtgaacttataccaatgcaatttcatatcaaacagtcacggatgggctttaggaaagcagaactgggcccggcccagtagatcacatagcccaacaagatctaaaccgatgctctcgtttcaacaaattatcacaccgattgcatttgcatctgctgcacaggctaaatcaaacacgtagccatgaaccattcacctcacaagtcacaagcattgcatttctatggttaccagtgcaggacgaaatgctcaactagcccaagcaagaatggagcatgacaacctcaggcaccaaaagctctataaatatttgcattgcacaaatcaaaagtttccaagttttaatcagagaagttcaagatatcagaaagaacaacacataccccgggatccgccatggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgatggcgccagcggctctgagactccaggcacctctgagtctgctaccccagagtctgacgcgctggacgatttcgacctggatatgctgggcagcgacgccctggatgacttcgatcttgacatgctcggctccgacgctctcgacgactttgacctcgacatgttgggctctgatgcccttgacgattttgatttggacatgctgagcggcagcgaaaccccaggcacttcagaaagcgctacgccagagagcgacgcgttggacgactttgatctcgatatgctggggtccgatgcgctcgatgacttcgacttggatatgttgggttctgacgctttggatgattttgaccttgacatgcttggtagcgacgcactcgatgattttgatctggatatgctctaggctca

SEQ ID NO: 21 - DNA, 12300 bp, synthetic construct
  misc_feature 1..12300: note = pBL- OsLPT6-tadCas9::VP128 M1
  source 1..12300: mol_type = other DNA
ctagttgatgagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaataagcttggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgtccggtttataaactcgcttgctgcatcagacttgcgggtgccgcactctcaacggttttagagctagaaatagcaagttaaaataaggctagtccgttatcaacttgaaaaagtggcaccgagtcggtgctttttttgcatgcaagctgatccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgcctttccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagatcttggcaggatatattgtggtgtaacgtatcacaagtttgtacaaaaaagcaggctccgcggccgcgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcctgcagggttccggcggtagtggaggtagtggtggcctccaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcaagcttgcatgcctgcaggtccccagattagcggcgcgccgaacctcagccaaaaccgaagacagtaccgccgaaggaccttatttaaacggttttgttaagttgggggacccatcgtacccggttttgcgaccggggacgaaaatcggactaggtgataaatagagggacccaaagtgaacttataccaatgcaatttcatatcaaacagtcacggatgggctttaggaaagcagaactgggcccggcccagtagatcacatagcccaacaagatctaaaccgatgctctcgtttcaacaaattatcacaccgattgcatttgcatctgctgcacaggctaaatcaaacacgtagccatgaaccattcacctcacaagtcacaagcattgcatttctatggttaccagtgcaggacgaaatgctcaactagcccaagcaagaatggagcatgacaacctcaggcaccaaaagctctataaatatttgcattgcacaaatcaaaagtttccaagttttaatcagagaagttcaagatatcagaaagaacaacacataccccgggatccgccatggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgatggcgccagcggctctgagactccaggcacctctgagtctgctaccccagagtctgacgcgctggacgatttcgacctggatatgctgggcagcgacgccctggatgacttcgatcttgacatgctcggctccgacgctctcgacgactttgacctcgacatgttgggctctgatgcccttgacgattttgatttggacatgctgagcggcagcgaaaccccaggcacttcagaaagcgctacgccagagagcgacgcgttggacgactttgatctcgatatgctggggtccgatgcgctcgatgacttcgacttggatatgttgggttctgacgctttggatgattttgaccttgacatgcttggtagcgacgcactcgatgattttgatctggatatgctctaggctca

SEQ ID NO: 22 - DNA, 4780 bp, synthetic construct
  misc_feature 1..4780: note = tadCas9::KRAB
  source 1..4780: mol_type = other DNA
atggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgatggcgccagcggctctgagactccaggcacctctgagtctatgaacaactcccaaggcagggtgaccttcgaggacgtgaccgtgaacttcacccaaggcgagtggcaaaggctcaacccagagcaacgcaacctctacagggacgtgatgctcgagaactactccaacctcgtgtccgtcggccaaggcgaaaccaccaagccagacgttatcctcaggctcgagcaaggcaaggaaccatggctcgaagaggaagaggttctcggctctggaagggccgagaagaacggcgatatcggcggccaaatctggaagccaaaggacgtgaaggaatccctctag

SEQ ID NO: 23 - DNA, 13731 bp, synthetic construct
  misc_feature 1..13731: note = pBL-Ubi-tadCas9::KRAB-sgRNA
  source 1..13731: mol_type = other DNA
ggccgcgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcctgcagggttccggcggtagtggaggtagtggtggcctccaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcaagcttgcatgcctgcaggtccccagattagcggcgcgccgtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgcccgggatccgccatggccccgaagaagaagaggaaggtgggcatggccccgaagaagaagaggaaggtgggcatggacaagaagtactccatcggcctggccatcgggaccaactctgtaggctgggcggtgatcacggacgagtataaggtgccctcgaagaagttcaaggtcctgggaaacaccgaccgtcacagcatcaagaagaacctgataggggccctcctgttcgactcaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtgagactgctgaggcgaccaggctgaagcgtactgcaagaagacgttacacgaggcggaagaacaggatctgctacctccaagagatcttcagcaacgagatggcgaaggtcgacgactcgttcttccaccgtctcgaggagagcttcctggtcgaggaagacaagaagcacgagaggcacccgatcttcgggaacatcgtggacgaagtggcgtaccacgagaagtaccccacaatctaccacctccggaagaagctggttgactccactgacaaggctgacctgcgtctcatctacctggccctcgcccacatgatcaagttcaggggtcacttcctcatagagggggacctgaaccctgacaatagcgacgtcgacaagctgttcatccagctggtgcaaacctacaaccagctcttcgaggagaaccccatcaacgctagtggagttgacgccaaggctatcctgtctgctaggctgtccaaaagccgacgcttggagaacttaatcgctcagcttccgggggagaagaagaacggactcttcgggaacctgattgccctgagtctgggcctgaccccgaacttcaaaagcaacttcgacctcgccgaggacgcgaaactccagctgtcaaaggacacatacgacgacgacctggacaacctgttagcacagatcggggaccagtacgccgacctctttctggcagctaagaacctgtctgacgccatcctcctgtccgacatcctccgagtcaacactgagatcaccaaggccccactgagcgcctctatgataaaacgctacgacgagcaccaccaggacctgactctcctgaaggctctcgtacgtcagcagctgcctgagaagtacaaggagatcttcttcgaccagtcgaagaacggctacgccgggtacattgatggcggggcttctcaagaggagttctacaagttcatcaagcccatcctcgagaagatggacggcaccgaggagctgctcgttaagttgaacagagaggacctgctccgaaagcagaggacctttgataacgggtcgatcccgcaccagatccatctcggtgagctccatgctatcctgcgacgccaagaggacttctaccccttcctcaaggacaaccgcgagaagattgagaagatcctgacattccgaatcccgtactacgtcgggcctcttgcaaggggcaacagccggttcgcttggatgacccgcaagtcggaggagactatcacaccctggaactttgaggaggtcgtggacaagggcgcatctgcccagagtttcatcgagaggatgaccaacttcgataagaacctcccgaacgagaaggtgctcccgaagcactctctgctctacgagtacttcaccgtgtacaacgagctgacgaaggtcaagtacgtgaccgagggtatgcggaagcctgcgttcctgagcggggagcaaaagaaggccatcgtcgatctcctgttcaagaccaaccgcaaggtgaccgtcaagcagctcaaggaggactacttcaagaagatcgagtgcttcgactccgtcgagatctcgggcgttgaggacaggttcaacgcaagcctggggacctaccatgacctgctcaagattatcaaggacaaggacttcctcgacaacgaggagaatgaggacattctagaggacatcgtgctcacgctgaccctcttcgaggaccgcgagatgatcgaggagcgcctcaagacctacgcccacctcttcgacgacaaggtgatgaagcaactgaagaggcgcaggtatactggatgggggaggctctcaaggaagctgatcaacggcatcagggacaagcagagcggcaagacgatcctcgacttcctgaagtctgacggcttcgctaaccgcaacttcatgcagctcatccacgacgactcgctcacgttcaaggaagacatccagaaggcgcaggtaagcggccagggtgattctctgcatgagcatatcgctaacctcgccggctcccccgcaatcaaaaagggaatcctccagaccgtcaaggtcgtggacgagctggttaaagtgatgggcaggcacaagcctgagaacatcgtgatcgagatggcgcgcgagaatcagacgactcagaagggccagaagaactcgcgcgagcgcatgaagagaattgaggagggtatcaaggaactcggcagccagatcctcaaggagcacccagttgagaacacccagctccagaatgagaagctgtacctctactacctgcagaacggcagggacatgtacgtggaccaagagctggacatcaacaggctgtccgactacgacgttgacgccatcgtgccccaaagtttcctcaaggacgactcgatcgacaacaaggtgctcacgaggtccgacaagaaccgcggcaagtctgacaacgtgccaagcgaggaggtcgtgaagaagatgaagaactactggcggcagctcctgaacgccaagctcatcacccagcgcaagttcgacaacttgacaaaggcagagagaggaggcctgtcggagcttgataaggcgggcttcattaagaggcagctcgtcgagactaggcagatcacgaagcacgtggcgcagatcctggatagcaggatgaacacgaagtacgacgagaacgacaagctcatccgcgaggtgaaggtcatcaccctcaagtccaagctggtgagcgacttccgcaaggatttccagttctacaaggtccgcgagatcaacaactaccaccacgcccacgacgcctacctgaacgcggtagttggcacggccctcatcaagaagtaccctaagctcgagagcgagttcgtgtacggcgactacaaggtgtacgacgtgcgcaagatgatcgcgaagtcggagcaggagatcggcaaggcgaccgccaagtacttcttctacagcaacatcatgaacttcttcaagaccgagatcacgctggccaacggcgagatcagaaagaggccgctcattgagactaatggtgagactggcgagatcgtgtgggataagggaagggacttcgcgactgtgcgcaaggttctgagtatgcctcaggtgaacatcgtcaagaagaccgaggtccagaccggcggcttcagcaaggagtcaatcctcccgaagaggaacagcgataagctgattgcgaggaagaaggactgggacccaaagaagtacggcgggttcgatagcccaaccgtggcttacagtgtgctggtggtggcgaaggtcgagaagggtaagtccaagaagctcaagtccgtgaaggagctgctcggcatcaccatcatggagcggtccagcttcgagaagaacccaatcgacttcctcgaggccaagggctataaggaggtcaagaaggacctcatcatcaagctgccaaagtactccctgttcgagcttgagaacggccgcaagaggatgctcgcttctgcaggagagttgcagaaaggcaacgagcttgcgctcccgtccaagtacgtcaacttcctgtacctcgccagccactacgagaagctgaagggctctccagaggacaatgagcagaagcagctattcgtcgagcagcacaagcactacctggacgagatcatcgagcagatcagcgagttcagcaagagggtgatcctcgccgacgcgaacctggataaggtgctctccgcctacaacaagcaccgcgacaagccaattcgcgaacaggccgaaaacattatacacctgtttacgctcacaaacctgggtgcgccagcagccttcaagtacttcgataccaccatcgaccgcaagcggtacacctccactaaggaggtgctcgacgcgaccctgatccaccagtctatcaccggtctctacgaaacaaggatcgacctgtctcagctcggaggtgatggcgccagcggctctgagactccaggcacctctgagtctatgaacaactcccaaggcagggtgaccttcgaggacgtgaccgtgaacttcacccaaggcgagtggcaaaggctcaacccagagcaacgcaacctctacagggacgtgatgctcgagaactactccaacctcgtgtccgtcggccaaggcgaaaccaccaagccagacgttatcctcaggctcgagcaaggcaaggaaccatggctcgaagaggaagaggttctcggctctggaagggccgagaagaacggcgatatcggcggccaaatctggaagccaaaggacgtgaaggaatccctctgagctcactagttgatgagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaataagcttggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgtccggtttataaactcgcttgctgcatcagacttgcgggtgccgcactctcaacggttttagagctagaaatagcaagttaaaataaggctagtccgttatcaacttgaaaaagtggcaccgagtcggtgctttttttgcatgcaagctgatccttgacaggatatattggcgggtaaactaagtcgctgtatgtgtttgtttgagatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaagaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgtgtaacattggtctagtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacaacattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcgatccccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatccccggaattagatcttggcaggatatattgtggtgtaacgtatcacaagtttgtacaaaaaagcaggctccgc

SEQ ID NO: 24 - DNA, 882 bp, synthetic construct
  misc_feature 1..882: note = Ms2 Bob White - cDNA
  source 1..882: mol_type = other DNA
atggcagggcaccacagcccctcggcggcctcggcactgcgtgaaaaagacacgctggtgaggtgtctcgtgggatcaggtcccggcggcggcgctcatgccgggaccttcggcgctgtgcgggacttcctcatccagttccgcgaccaaggaatcccctgggtccgcatctacgagtcaaccccggcttggcagcagcaatccggcgggctgctgatccaggattgggacggagacgccgcggcggagggagccaaggtgttcttcacgctcatcaccacaaggaggggcggcgccattaacaggagggcactgggaggcgggacgtggacaagcaaggccgcgcccagggtaggggacgaggtcgccgtcagcacactgtacttcaaacggggcgggtccagcggcagattattcaccgccttggagatccatctcagaaacgagccccaagttgctatctgcctgctgcatccgactaactatctgtatagcattcgggatttgaggctctacatcgaccagggatggttcccgggaggaactcaagcaaacctgggcgcggagcaatatcaagatcctgatgttcctggattcgtgagtggttcacgtgctgattacaccactattctgttttctagcagtgagactatttacgaccagcaatcgattcattcctccggggctgctctgccacctcatgatgcatctctggatgctatttctcaccacctgttttcagaaaacaactcaacgccagagtttggtggacagtattctcatgctgatgaaatatcaatccttaatgaatactacaataccttgatggggaccaactccaactcaggattgcatgccttatccgcagcattctcaagttga

SEQ ID NO: 25 - DNA, 236 bp, synthetic construct
  misc_feature 1..236: note = Part of MS2 without intron
  source 1..236: mol_type = other DNA
atggcagggcaccacagcccctcggcggcctcggcactgcgtgaaaaagacacgctggtgaggtgtctcgtgggatcaggtcccggcggcggcgctcatgccgggaccttcggcgctgtgcgggacttcctcatccagttccgcgaccaaggaatcccctgggtccgcatctacgagtcaaccccggcttggcagcagcaatccggcgggctgctgatccaggattgggacggaga

SEQ ID NO: 26 - DNA, 554 bp, synthetic construct
  misc_feature 1..554: note = Ms2 Bob White - gene
  source 1..554: mol_type = other DNA
atggcagggcaccacagcccctcggcggcctcggcactgcgtgaaaaagacacgctggtgaggtgtctcgtgggatcaggtcccggcggcggcgctcatgccgggaccttcggcgctgtgcgggacttcctcatccagttccgcgaccaaggaatcccctgggtccgcatctacgagtcaaccccggcttggcagcagcaatgtgagtaatctaatctccactgttgattgatctgcataatgctactcattctcactatcgcctggccggcctcgtgatcaacatgaatgcgcgcgtatgttatgcagccggcgggctgctgatccaggattgggacggagacgccgcggcggagggagccaaggtgttcttcacgctcatcaccacaaggaggggcggcgccattaacaggagggcactgggaggcgggacgtggacaagcaaggccgcgcccagggtaggggacgaggtcgccgtcagcacactgtacttcaaacggggcgggtccagcggcagattattcaccgccttggagatccatctcagaaacgag

SEQ ID NO: 27 - DNA, 20 bp, synthetic construct
  misc_feature 1..20: note = primer
  source 1..20: mol_type = other DNA
actgtctcgcgatttgagca

SEQ ID NO: 28 - DNA, 20 bp, synthetic construct
  misc_feature 1..20: note = primer
  source 1..20: mol_type = other DNA
tctccgtcccaatcctggat

SEQ ID NO: 29 - DNA, 20 bp, synthetic construct
  misc_feature 1..20: note = Primer Cas9endfw1
  source 1..20: mol_type = other DNA
catcgagcagatcagcgagt

SEQ ID NO: 30 - DNA, 20 bp, synthetic construct
  misc_feature 1..20: note = Primer U6promrw1
  source 1..20: mol_type = other DNA
gctctccccaatcgtgaaca

(19) United States
(12) Patent Application Publication
(10) Pub. No.: US 2025/0051791 A1
(43) Pub. Date: Feb. 13, 2025

(54) A SYNTHETIC GENE EXPRESSION REGULATOR AND A PROCESS FOR PRODUCING HYBRID CROPS USING THEREOF

(71) Applicant: SZKOLA GLOWNA GOSPODARSTWA WIEJSKIEGO W WARSZAWIE, Warszawa (PL)

(72) Inventors: Leszek Aleksander LYZNIK, Palm City, FL (US); Monika RAKOCZY-TROJANOWSKA, Warszawa (PL); Magdalena SWIECICKA, Warszawa (PL); Bartosz SZABALA, Leszno (PL)

(21) Appl. No.: 18/701,455

(22) PCT Filed: Oct. 24, 2022
(86) PCT No.: PCT/PL2022/050069

§ 371 Date: Apr. 15, 2024

(30) Foreign Application Priority Data
Oct. 22, 2021 (PL) P.439284

(51) Int. Cl. C12N 15/82; C12Q 1/6844; C12Q 1/6895

(52) U.S. Cl. CPC C12N 15/8231; C12Q 1/6844; C12Q 1/6895; C12Q 2600/13

(57) ABSTRACT

A synthetic expression regulator for a plant gene is disclosed, related to a phenotypic trait of male sterility, and a process for producing male sterile plants using synthetic regulators and use thereof for breeding hybrid crop varieties.

### FIELD OF THE INVENTION

The invention concerns a process for producing male sterile plants using synthetic regulators and their use for breeding heterotic crop varieties. Male sterility systems are the mainstay of heterotic plant breeding programs. The effect of heterosis enhances crop productivity, which is particularly manifested when breeding corn and rice hybrids. One of the tenets of plant breeding is an assumption that yields of hybrid (heterotic) crops are higher than when cultivating homozygous inbred plants. The cultivation of hybrids is the basis for corn production worldwide. According to estimates, one-fifth of all rice crops are currently hybrid varieties introduced by Yuan Longping in the 1970s. Rice yields increased by approx. 30% have ensured food for millions of people, particularly in Africa and Asia. In addition, heterotic plant varieties have some other valuable traits, such as stable yield and higher tolerance to environmental stressors.

### SEQUENCE LISTING

The instant application contains a Sequence Listing which has been submitted electronically in XML file format and is hereby incorporated by reference in its entirety. Said XML copy, created on Jun. 7, 2024, is named sequence.xml and is 123,447 bytes in size.

### BACKGROUND OF THE INVENTION

The few registered and currently cultivated heterotic wheat hybrids (e.g., Hybred F1, Hy-mack F1, Hystar F1, Hymalaya F1, Hyvega F1 from Saaten-Union GmbH) typically have yields 3-15% higher compared to conventional varieties. Therefore, development of such varieties is highly justified and expected by breeders and farmers, seed material distributors, and the crop industry. Heterotic wheat varieties may be cultivated on poorer soils and in the low input farming system which prevents excessive environmental pollution, because they typically use elements, including nitrogen, with greater efficiency.

The lack of effective production methods for hybrids of other grain crops, such as wheat and barley, significantly limits the progress of breeding efforts and grain production from these crops. (1, 2).

Hybrids with Monoecious inflorescences (corn) are obtained by mechanically removing male inflorescences from parent plants. This cannot be achieved with plants having dioecious inflorescences, in particular cleistogamous plants (pollination occurs before flower opening) that are self-pollinating (rice, wheat, barley, etc.). A finding is used in this group of plants that mitochondrial damage may result in the sterility of maternal plants (cytoplasmic male sterility). When they are crossbred with plants having a trait that restores fertility (restorers), fertile hybrids form. However, the method is not without disadvantages. Damaged mitochondria reduce plant resistance to external factors, and the fertility restoration process may not be completely effective. An alternative strategy is to destroy pollen chemically to obtain sterility. However, this method is technically complex and because it involves the use of aggressive chemicals, it is harmful to the environment. Therefore, there still is a need for an improved process for producing hybrids usable in plant breeding programs for agricultural production.

Male sterility is a genetic trait observed in plants, critical in production systems for plant hybrids with dioecious inflorescences. Mutations mainly involve genes responsible for the growth of pollen grains or anthers. These are primarily recessive genetic traits that require the production of homozygous mutated plants to generate parent forms(3). Such plants do not produce pollen and, therefore, they can hardly be propagated as homozygous inbred lines. In addition, the recessive trait of sterility is difficult to achieve and maintain in polyploid plants, such as wheat(4), whose genomes have several copies of mutated genes.

Complex breeding systems have been developed that use recessive sterility genes to produce corn hybrids, such as the SPT platform(5). The SPT system uses recessive sterility mutations and mutated plants are propagated through genetic transformations with a fertility restorer gene. The presence of the gene is monitored by marker genes, and sterile plants are isolated by sorting. In addition, pollen grains containing a fertility gene are destroyed in the sterile plant propagation process through the expression of a fused α-amylase gene. However, the use of the system in plants other than corn (such as in hexaploid wheat plants) seems to be very complex and problematic.

Breeding technology based on dominant sterility genes may open up new opportunities for breeding heterotic plants, in particular those with complex genomes(2). However, only a few dominant sterility genes are known. For example, an MS2 dominant sterility gene was identified in wheat in 1972(6). It is a product of a random natural mutation. This gene was successfully coupled with a phenotypic trait of dwarfing, and thus a system for producing hybrid plants was developed. When used in practice, wheat hybrids showing increased yields were obtained. However, the breeding process was complex and required multi-generational breeding and selection.

The objective of this invention is to provide universal technology to enable the relatively simple production of male sterile plants useful for obtaining hybrid plants. The basis for the present invention is the use of synthetic transcription regulators of genes that determine phenotypic traits of male sterility in grain crops.

Synthetic transcription regulators (SRTs) are known. They are commonly used for model studies(8), but their practical uses were limited due to the lack of flexibility in binding to and regulating activity of natural promoters. The attachment of the Cas9 protein to natural activators made it possible to expand the applications of synthetic activators(9). The VP-64 viral activation domain and the KRAB repressor domain are popular components of synthetic activators; however, some other options are available(9-11).

Another objective of the invention is to provide a process to produce crop hybrids. The current production methods for grain crop hybrids using nuclear fertility genes (GMS) are based on their mutations leading to the formation of a male sterility trait, followed by restoration of fertility by introducing extra copies of fertility genes. In such a genetic environment, the extra copies of fertility genes become a dominant trait that can subsequently be identified.

The core of the invention is related to the use of a synthetic expression regulator for fertility genes as a genetic agent that leads to the formation of a dominant male sterility trait. Therefore, fertility genes are not permanently modified (mutated) in any way, and restoration of fertility involves desegregation (removal) of the synthetic regulator in the offspring (seeds) of parent plants.

### SUMMARY OF THE INVENTION

The invention concerns a synthetic gene expression regulator for crops and a process for producing hybrid crops using thereof as defined in detail in the attached claims. The first object of the invention is an expression regulator of a plant gene related to a phenotypic trait of male sterility being a protein-RNA complex wherein the protein has a protein DNA binding domain and a promoter activator or repressor domain for the transcription regulation of the gene linked with the former, wherein the protein DNA binding domain forms a complex with an RNA molecule having a sequence complementary to the element that controls expression of the sterility gene.

In the context of the present invention, the “gene related to a phenotypic trait of male sterility” is understood as a gene encoded by DNA found in the cell nucleus whose product leads to the abnormal development of microspores and elimination of pollen grains or leads to the abnormal development of anthers, that is, structures in which pollen grains develop. The genes are abbreviated as GMS (“genic male sterility”) (12).

The examples of known plant genes related to a phenotypic trait of male sterility that may be used according to the present invention are ZmMs7, ZmMs20, ZmMs30, and ZmMs26 in corn (12, 13, 28), Ta Ms1, TaMs2 in wheat (7, 14), TaNP1 in wheat or OsNP1 in rice and ZmIPE1 in corn(4). More than 100 GMS genes are known in plants, and a prerequisite for their usability in the present invention is knowledge about the structure and sequence of the elements that control their expression.

In the context of the present invention, the “protein DNA binding domain” is understood as a peptide, oligopeptide, or polypeptide molecule having a binding property with a DNA molecule having a specific DNA sequence.

The examples of known “protein DNA binding domains” that may be used according to the present invention are: DNA binding domains of transcription factors known as zinc fingers (15, 16), protein DNA binding domains of transcription activators known as TAL effectors(17), synthetic DNA binding domains of endonucleases, such as I-Crel (13), non-active endonucleases or their fragments that specifically bind DNA through complexes with RNA molecules, such as Cas proteins of the CRISPR system(18, 19). In the context of the present invention, the “RNA molecule having a sequence complementary to the element that controls expression of the sterility gene” is understood as an oligo—or polyribonucleotide whose part or the whole sequence is complementary to sequences found in promoters whose activity is regulated by the synthetic regulator. The examples of known “RNA molecules having a sequence complementary to the element that controls expression of the sterility gene” that may be used according to the present invention are RNA molecules known as tracrRNA and crRNA (20) or their synthetic versions known as sgRNA being components of the CRISPR system(21).

In the context of the present invention, the “protein activator domain” is understood as a peptide, oligopeptide, or polypeptide molecule whose presence in the promoter region leads to the activation of the gene whose expression is controlled by the promoter. The examples of known “protein activator domains” that may be used according to the present invention are natural domains of transcription factors, such as VP-16 and its synthetic derivatives (SunTag, TV) (9, 22).

In the context of the present invention, the “protein repressor domain” is understood as a peptide, oligopeptide, or polypeptide molecule whose presence in the promoter region leads to the inactivation of the gene whose expression is controlled by the promoter. The examples of known “protein repressor domains” that may be used according to the present invention are natural repressor domains, such as JAZ (23), ZTC1 (24), SRDX (11, 25), KRAB (“Kruppel-associated box”) repressor domains (26) or prokaryotic repressor domains, for example, TetRs (tetracycline-family repressors”) (27).

### DETAILS OF THE INVENTION

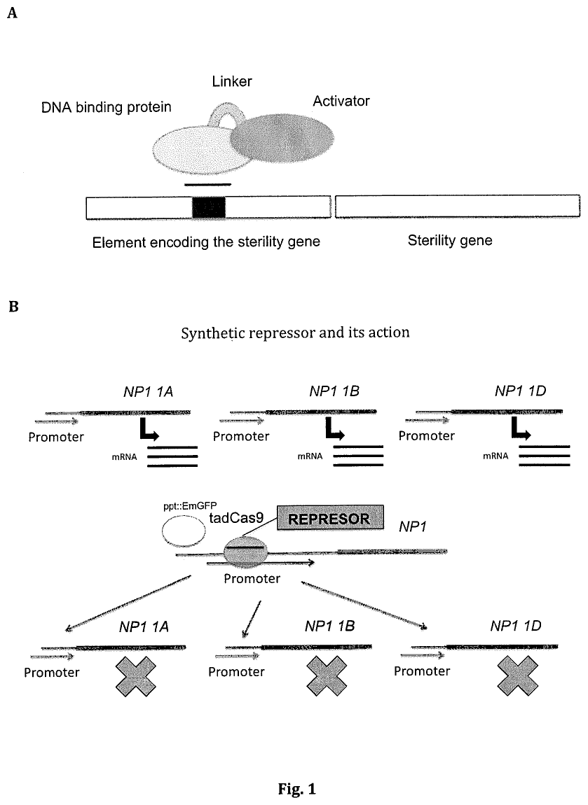

To enable a better understanding of the gist of the invention, it is additionally illustrated in FIG. 1. The genetic element that determines male sterility is an endogenous sterility gene whose product is a gametocide leading to the atrophy of male spores (microspores). Its activity is intrinsically limited by the control element in fertile plants. The control element is an endogenous inactive promoter of the sterility gene. By introducing a synthetic expression regulator of the sterility gene into the cell and its fusion with the control element, expression is activated by the activator found in the synthetic regulator, leading to the production of male sterile plants. The presence of the synthetic regulator is a dominant trait, and it may control the expression of several genes related to the phenotypic trait of sterility. In a preferred embodiment, the synthetic regulator may also be designed to stop the expression of recessive sterility genes and sterile plants are thus produced. In such a variant of the invention, the synthetic regulator is a synthetic repressor known as a genic dominant trait.

Cas endonuclease proteins (elements of the CRISPR system) can recognize specific sequences in genomic DNA. This is a feature determined by RNA molecules. According to the invention, a Cas9 endonuclease protein was used as the DNA-binding protein found in the synthetic regulator. By attaching a protein activator or repressor domain to the DNA-binding protein using a peptide linker, a synthetic regulator structure was obtained that enabled the function of specific genes to be switched on or off. The resulting synthetic regulator of gene expression can be used to model the activity of genes responsible for producing spores, in particular microspores, in plants.

The object of the invention is a process for producing crop hybrids using a synthetic regulator of gene expression. The synthetic regulator used according to the invention is a protein-RNA complex. The synthetic regulator designed according to the invention includes a protein domain being a DNA binding protein and an activator domain with the ability to activate gene expression in the activator binding site or a repressor domain with the ability to silence gene expression in the repressor binding site. In a preferred embodiment of the invention, the specificity of DNA binding by regulator complexes is determined by the RNA molecules that form a complex with the DNA binding protein.

The synthetic regulator is not a sterility gene. It serves as a genetic switch. According to the invention, the synthetic regulator may be considered a dominant, non-recessive genetic agent that determines the trait of sterility. This is a dominant trait even when the synthetic repressor acts on the recessive genes of male sterility. Abnormal regulation of gene expression in male sterility genes may lead to the production of sterile plants(28).

### Example 1. A Synthetic Gene Expression Regulator Containing an Activator Domain

FIG. 1 shows schematically the structure of the synthetic regulator having a role of a synthetic activator. The synthetic regulator consists of three elements: a domain that targets the activator to the activation site (DNA binding protein), a domain that causes activation of the expression of gene(s) that induce male sterility (activator) and an element that combines the two domains in one protein (linker). The elements of the synthetic regulator are known to molecular geneticists separately and there is much evidence that they are effective in other applications(29).

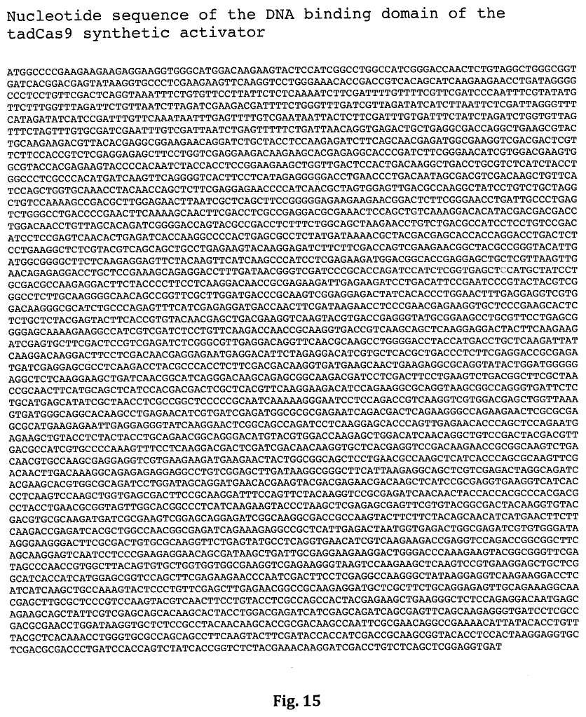

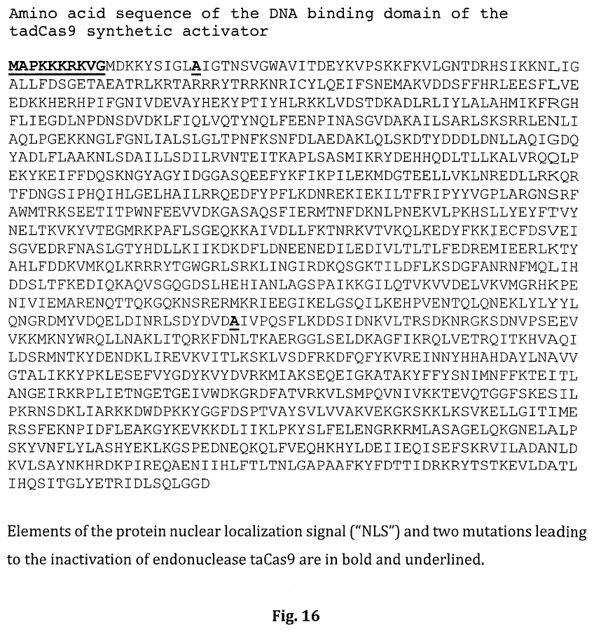

In the presented embodiment of the invention, a non-active tadCas9 endonuclease protein was used as the DNA binding protein, but other known proteins that specifically bind DNA sequences can be used according to the invention. The nucleotide sequence of the tadCas9 encoding gene is shown in FIG. 15 (SEQ ID No. 1), and its amino acid sequence is shown in FIG. 16 (SEQ ID No. 2). The tadCas9 protein contains two endonucleolytic domains (RuvC and HNH). Both domains were inactivated in the synthetic regulator by replacing the aspartic acid codon at position 20 (GAC) with an alanine codon (GCC) (RuvC domain) and replacing the histidine codon at position 850 (CAT) with an alanine codon (GCC) (HNH domain) (FIG. 16). The changes have no effect on the protein ability to bind DNA, and they eliminate the enzymatic activity of endonucleases whose activity is not desirable in the synthetic activator(30).

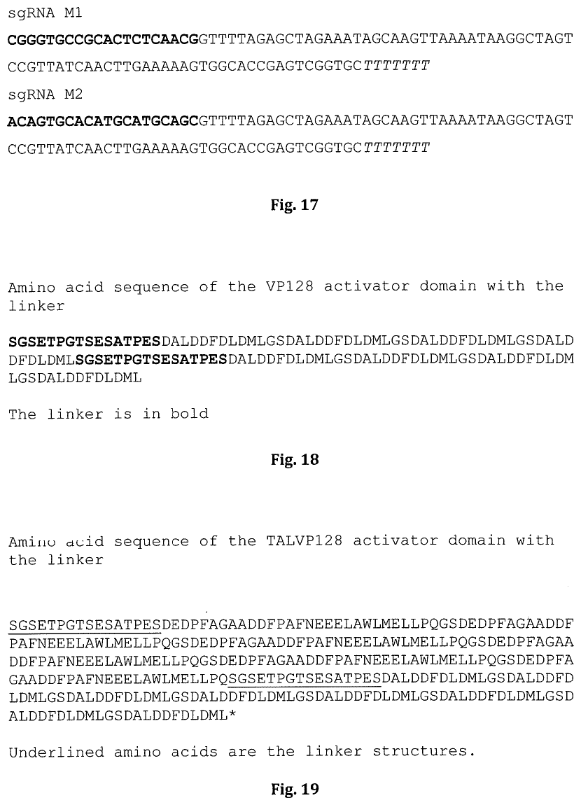

In the present embodiment of the invention, the binding specificity of the synthetic activator to the DNA sequence is obtained via sgRNA molecules introduced to plant cells with the synthetic activator in a way illustrated in Example 10. The sequences of example RNA molecules also known as sgRNA are shown in FIG. 17 (SEQ ID No. 3 and SEQ ID No. 4). The sequences complementary to the two binding sites of the synthetic activator to the promoter of the MS2 gene (M1 and M2) are denoted in bold. The other sequences denote the transcription end of the sgRNA molecule (TTTTTTT) and the structural element that enables the formation of a complex with the tadCas9 protein.

In an example embodiment, the fusion of the DNA binding domain (tadCas9) with the VP128 activator domain (tadCas9::VP128) is the structural basis for the synthetic regulator. The VP128 activator domain consists of eight repeats of the 5′-terminal activator domain of the HSV transcription factor(31). The amino acid sequence of the activator domain is shown in FIG. 18 (SEQ ID No. 5).

The primary activator unit is the activation domain of the VP16 viral activator (DALDDFDLDML). In a natural activator, it is in the 5′-terminal 81-amino acid part of the activator; however, it has been found that an eleven amino acid peptide is sufficient to maintain the activator function. Its four repeats are separated with a GS dipeptide from a VP64 activator whose activity has already been tested in plants(32). In the presented synthetic regulator (SEQ ID No. 5) the VP64 element is repeated and separated by two linkers (FIG. 18)(9, 33). The coding sequences of the activator and linkers have been optimized for expression in wheat cells.

The VP128 activation domain is a modular structure that may be modified to enhance its activity. The TV activation domain is an existing example of such activators(9). It contains six copies of the TAL activator and two copies of the VP64 activator. A variant of the synthetic activator designed for the activation of promoters in monocotyledonous plants (in wheat) is shown in FIG. 19 (SEQ ID No. 6).

### Example 2. Male Sterility—Inducing Gene and its Activation

Any fragment of genomic DNA whose expression leads to the formation of a phenotypic trait of sterility can be activated by a genetic sterility factor. In an example embodiment of the invention, a fragment of genomic DNA from the wheat genome was used, known as MS2. This genomic fragment does not have annotated any specific function and it is not expressed in wheat plants(7, 34).

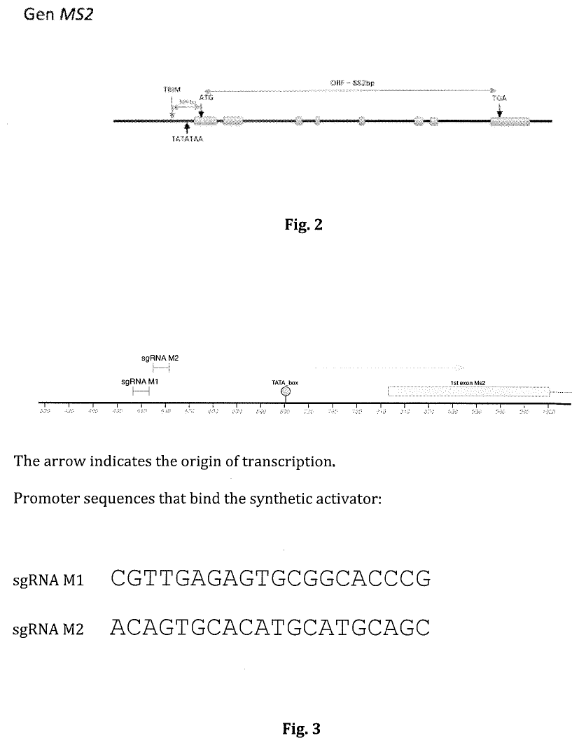

The MS2 gene is located on the short arm of chromosome 4 D in wheat cells. It consists of eight exons and seven introns (FIG. 2). Its homologs in subgenomes A and B are structurally mutated. The MS2 gene is inactive in fertile wheat plants, even though its promoter contains structural elements of fully functional promoters (TATA and CAT elements). Activation of the gene through a natural mutation (integration of retrotransposon TRIM in the promoter sequence) leads to the formation of male sterile plants(34). The MS2 promoter sequence in BobWhite wheat is shown in FIG. 25 (SEQ ID No. 16).

The MS2 promoter is used as an example to illustrate the activation of expression by the synthetic regulators of the invention. A pair of primers (ACTGTCTCGCGATTTGAGCA (SEQ ID No. 27) and TCTCCGTCCCAATCCTGGAT (SEQ ID No. 28)) was used to amplify the genomic fragment (1142 base pairs) containing the MS2 gene promoter. Based on the MS2 promoter sequences, sgRNAM1 and sgRNAM2 molecules (FIG. 17) were designed that target the synthetic regulator to the MS2 promoter sequences at approx. 200 base pairs from the origin of transcription. The binding sites for the DNA binding protein comprised in the synthetic regulator (M1 and M2 sequences) are shown in FIG. 3. Even though RNA molecules do not constitute an integral part of the synthetic regulator, their design and structure are the basis for the adequate function of the synthetic regulator in the present embodiment.

The RNA molecules may be omitted when other types of DNA binding domains are used in the synthetic regulators of the invention.

### Example 3. A Vector that Encodes Elements of the Synthetic Regulator Containing the Activator Domain

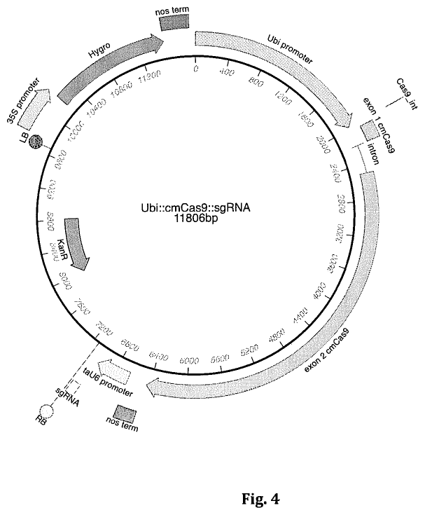

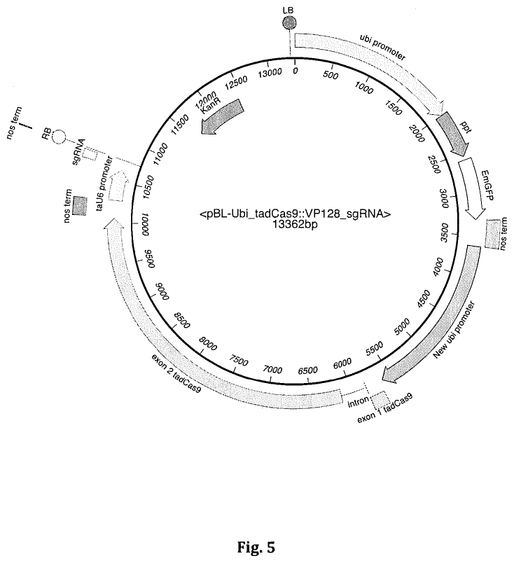

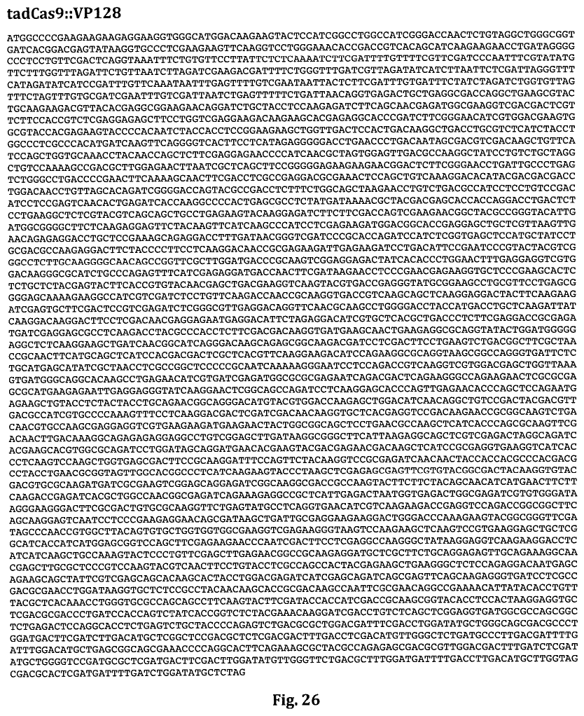

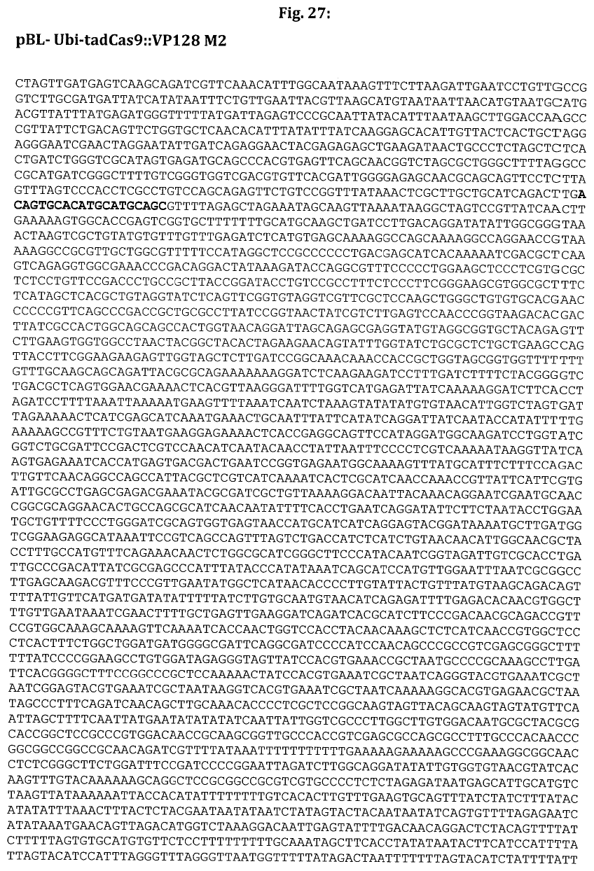

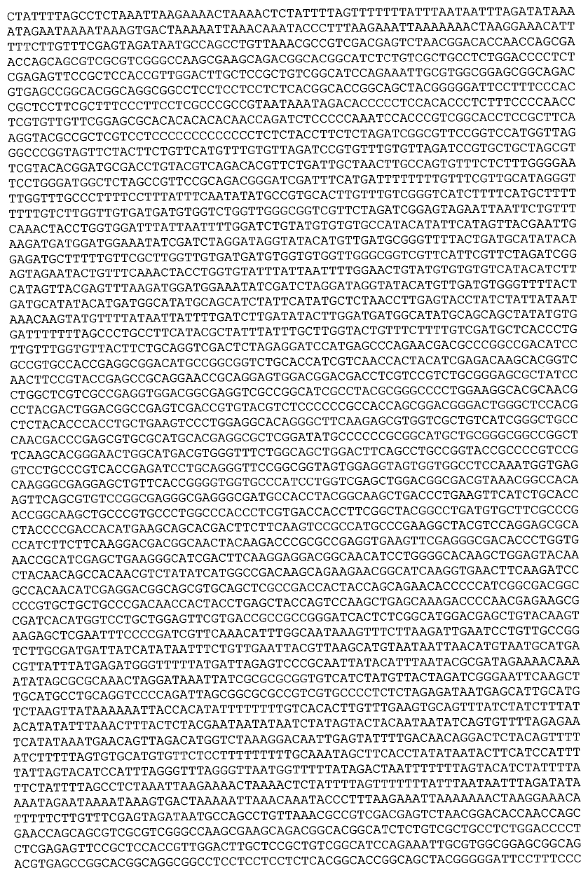

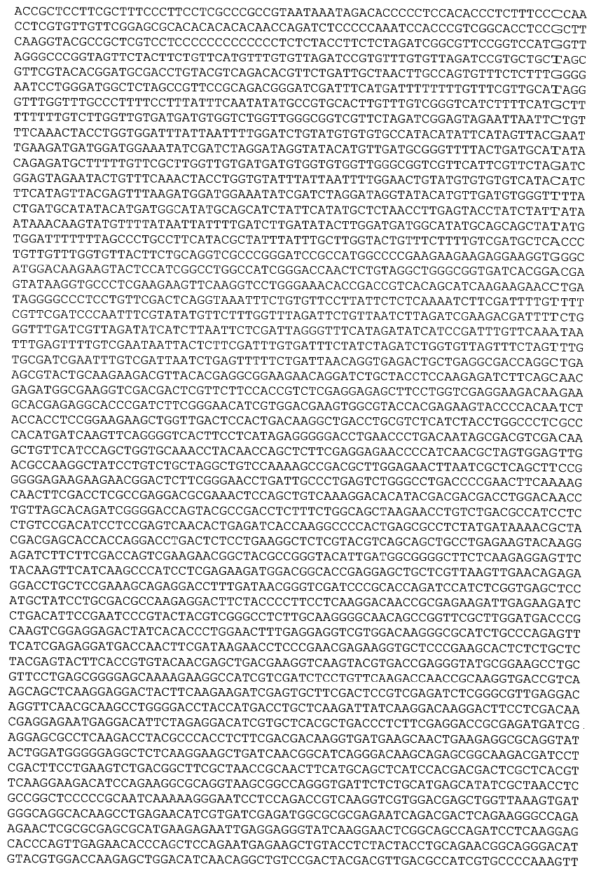

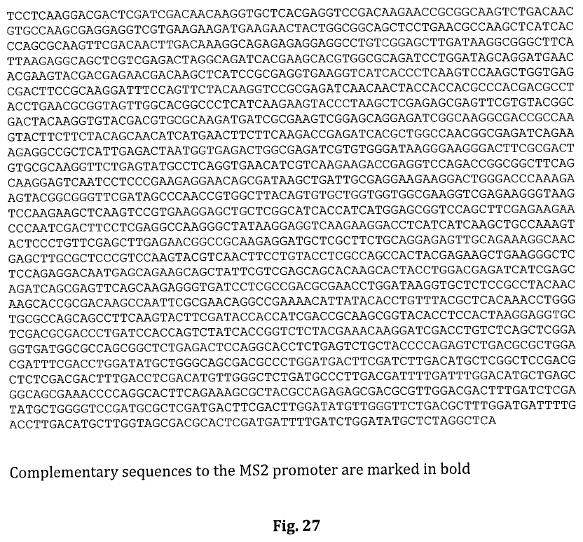

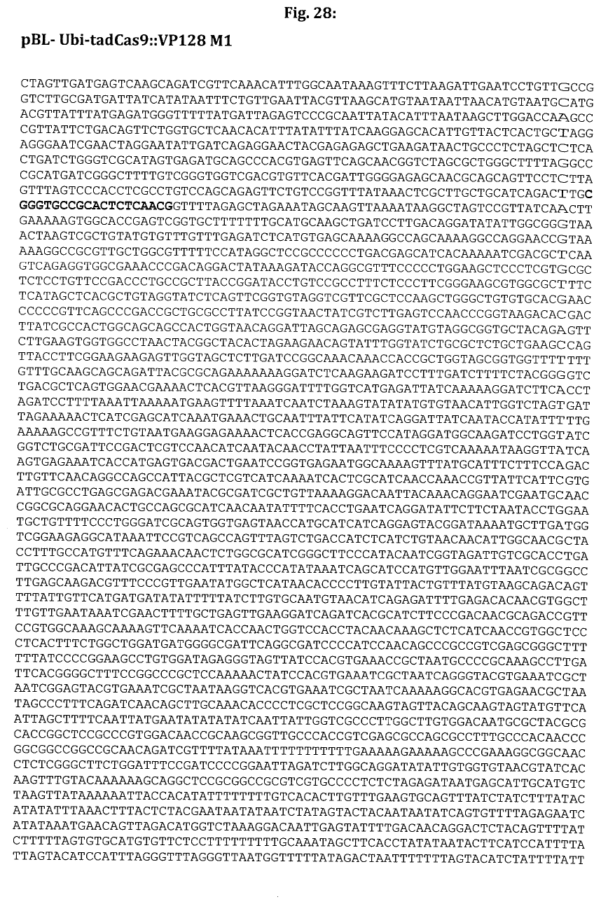

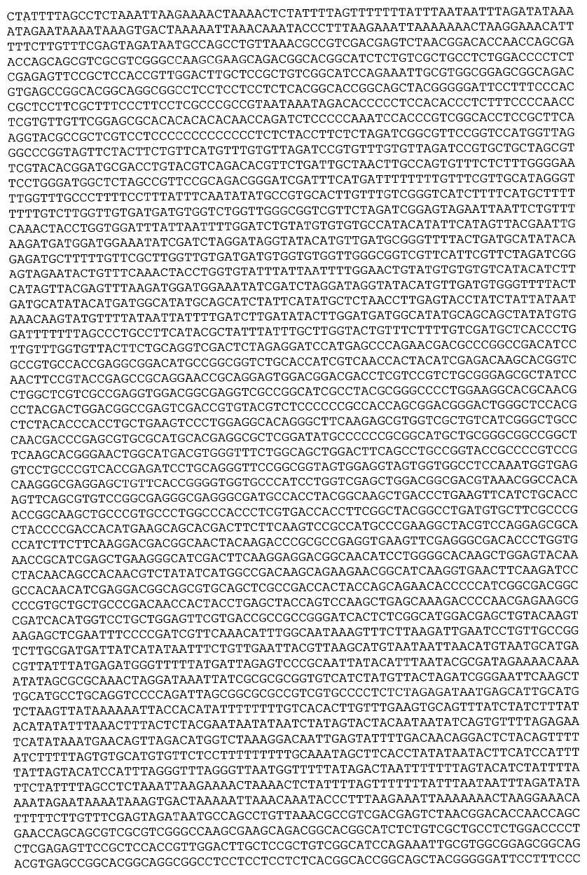

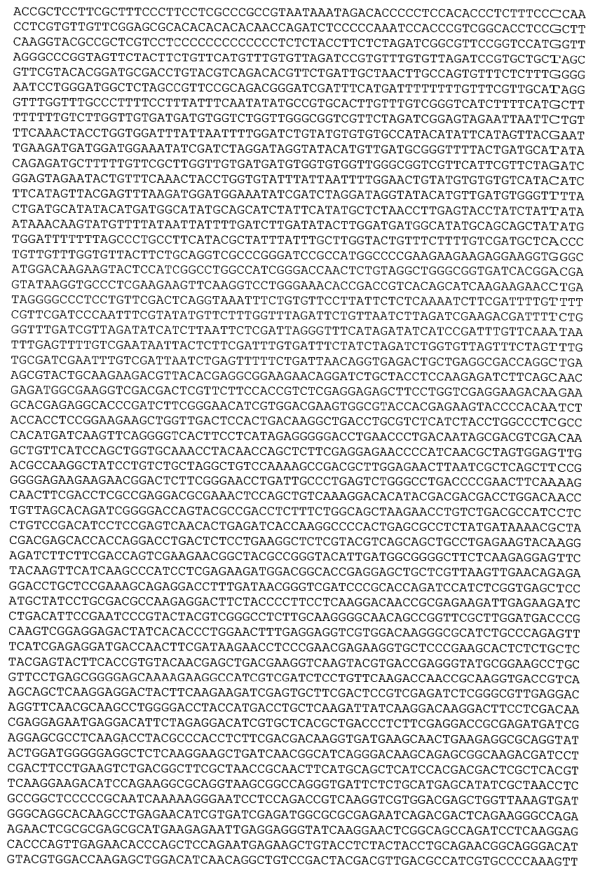

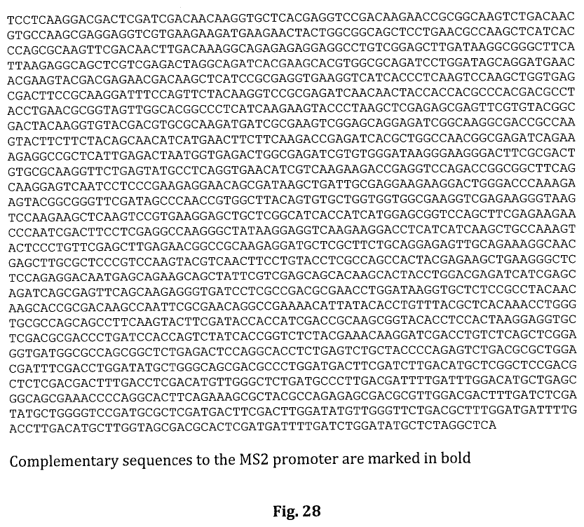

Methods for the synthesis of vectors to incorporate foreign genetic elements into cells of eukaryotic organisms are well-known by geneticists and molecular biologists. In an example embodiment of the invention, DNA vectors were used, based on known vectors termed pBract211 (John Innes Centre), and in particular, the pBract211-cmCas9-sgRNA vector shown in FIG. 4(35). The coding sequence of the cmCas9-int gene was replaced with a coding sequence of a synthetic activator, tadCas9::VP128 (SEQ ID No. 17, FIG. 26). The taU6-sgRNA cassette was modified to increase the yield of sgRNA molecules. The 35S-Hyg-int selection gene was replaced with an original ppt-EmGFP selection gene having a double function (visual and chemical selection marker). In addition, the p-Ubi promoter was replaced with a different variant having an altered structure. The vector designed and obtained in this way is denoted by the acronym pBL. The necessary genetic elements for the example embodiment of the invention and their location in the pBL vector molecule are shown in FIG. 5 (see also FIGS. 27 and 28 and SEQ ID No. 18 and 19).

Cloning of 20 nucleotide sequences complementary to the binding sites of the synthetic activator shown in FIG. 17 between two Bsal restriction sites of the pBL-Ubi_tadCas9::VP128_sgRNA vector leads to the formation of functional synthetic activators of the MS2 wheat gene: pBL-Ubi_tadCas9::VP128_sgRNA M1 (SEQ ID No. 18) and pBL-Ubi_tadCas9::VP128_sgRNA M2 (SEQ ID No. 19)

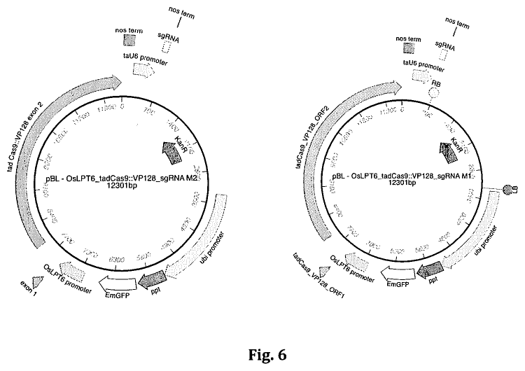

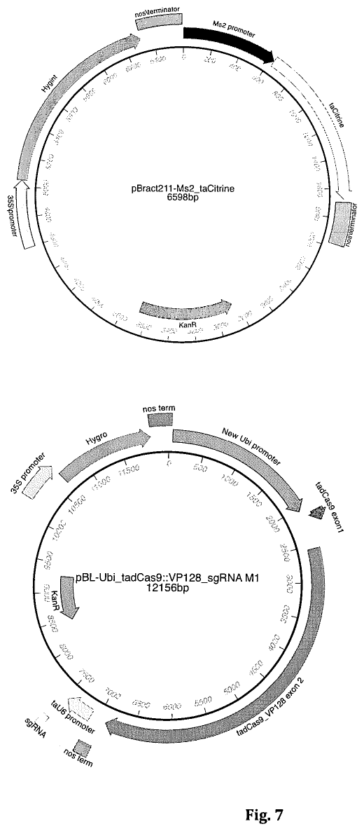

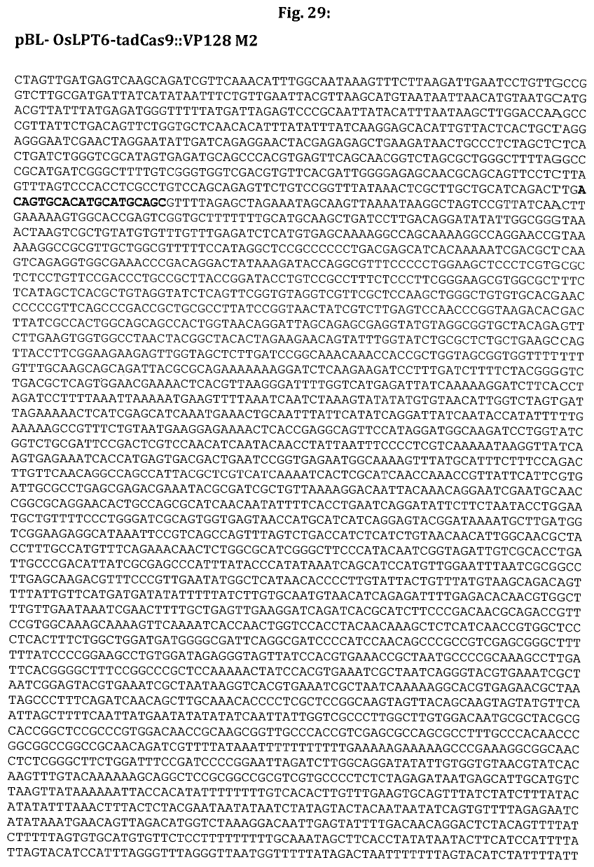

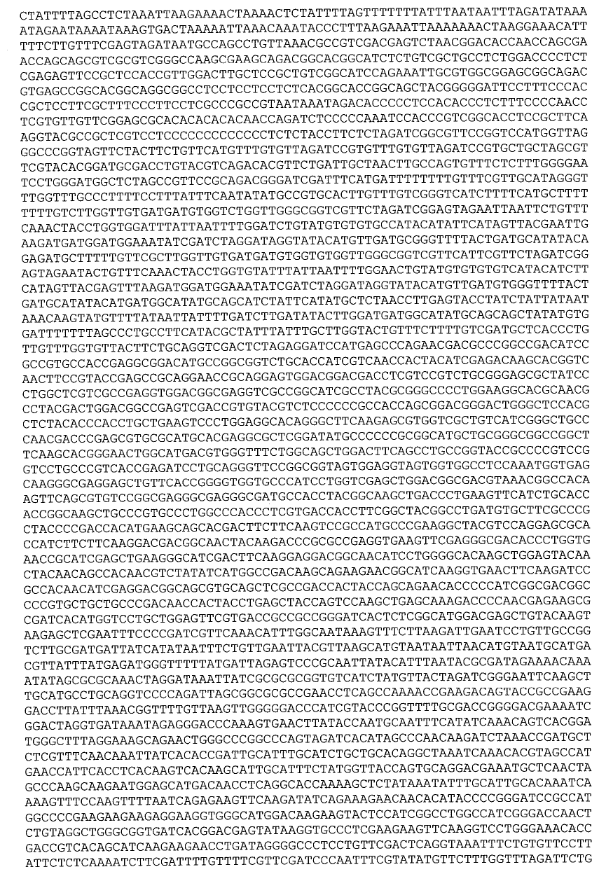

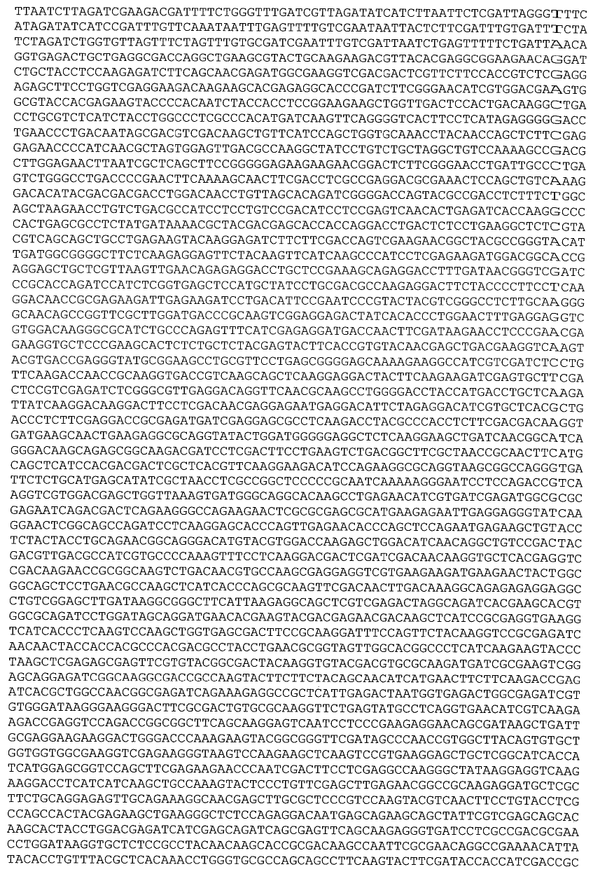

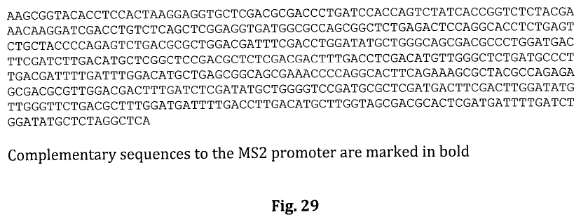

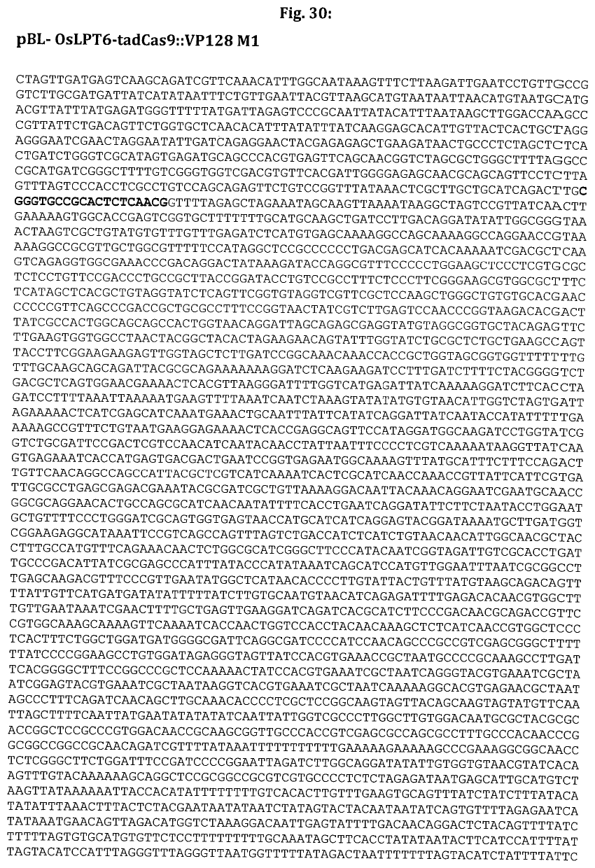

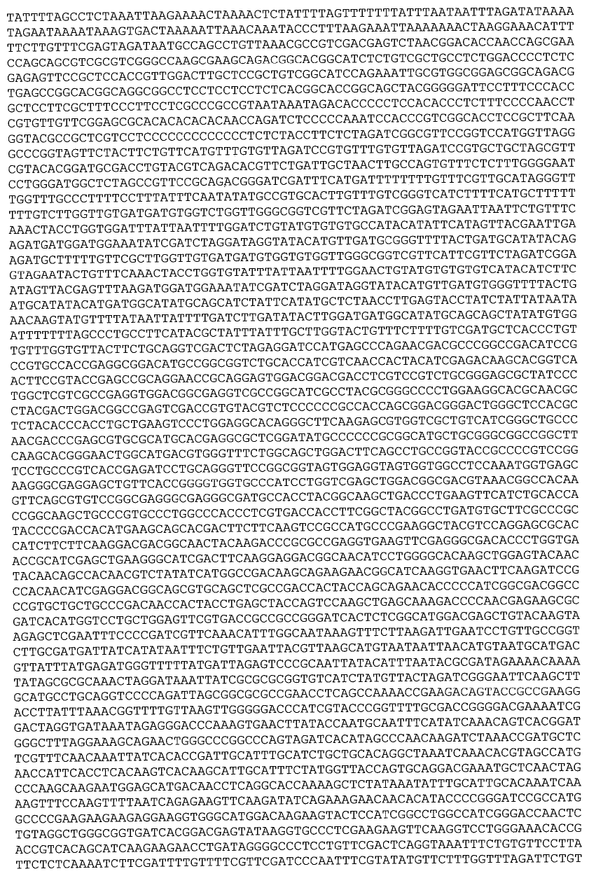

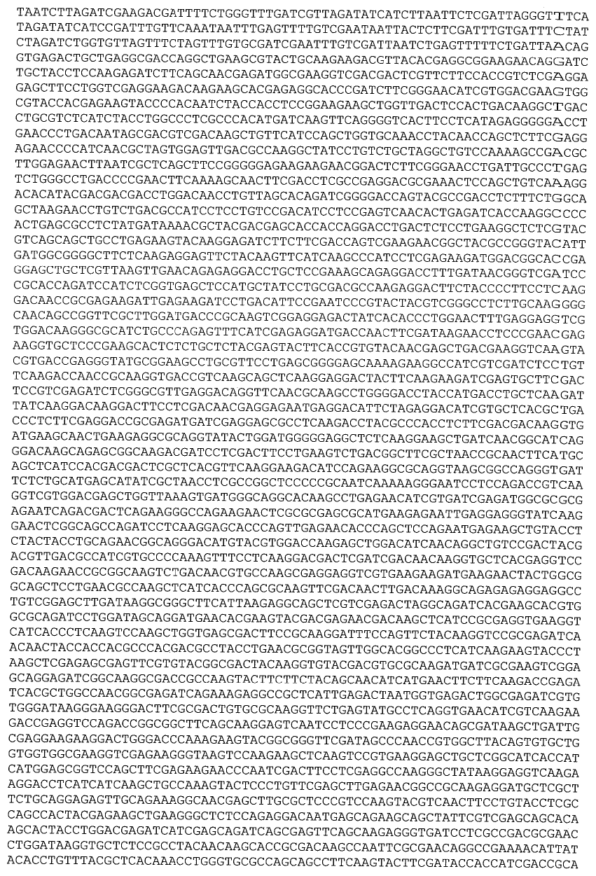

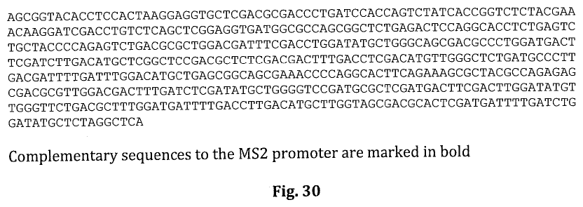

Synthetic regulators may be targeted to specific tissues or organs by the control of their expression and accumulation. In an example embodiment of the invention, the promoter that controls the expression of the synthetic activator was replaced with a specific rice promoter active in anthers (OsLTP6)(36). The two vectors (pBL-OsLPT6_tadCas9::VP128_sgRNA M2 and pBL-OsLPT6_tadCas9::VP128_sgRNA M1) are shown in FIG. 6 (see also FIGS. 29 and 30 and SEQ ID No. 20 and 21).

### Example 4. A Synthetic Gene Expression Regulator Containing a Repressor Domain

Transcription factors containing repressor domains are common in living organisms. More than 700 repressors having structures of zinc fingers are known in the human genome, of which approx. 300 contain a repressor domain known as the “Kruppel-associated box” (KRAB) (16). These are polypeptides of approx. 50-70 amino acids divided into two segments: “boxA” and “boxB”. Their functionality in combination with the Cas9 protein domain was tested; the ZIM3 KRAB domain was found to be one of the best KRAB domains in human cells(26).

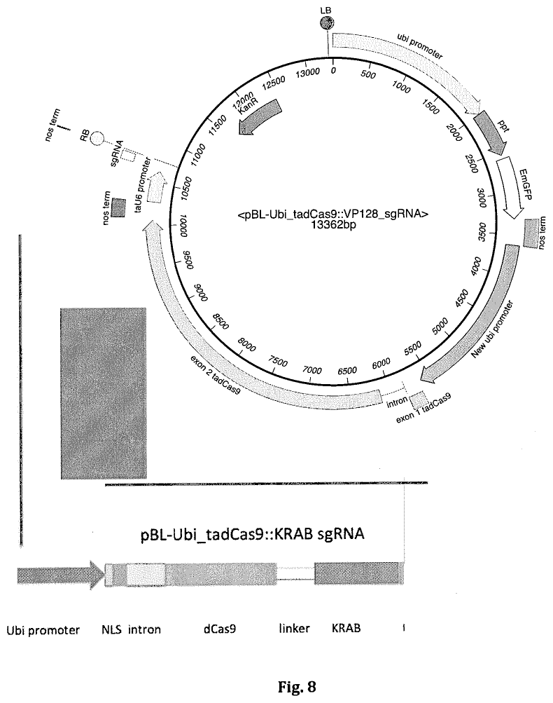

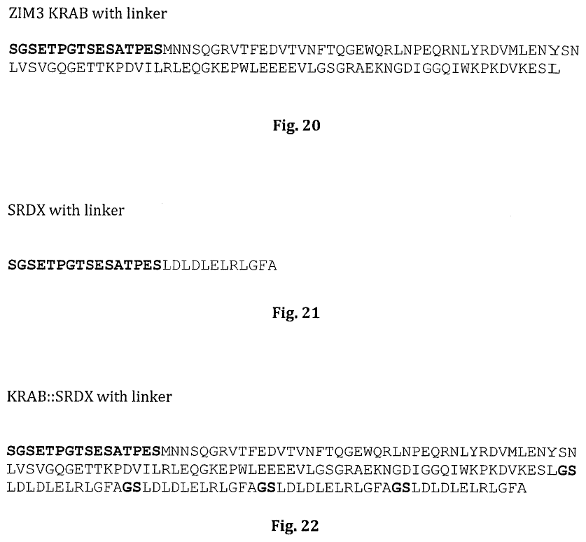

In the present embodiment of the invention, the synthetic regulator that limits the expression of a plant gene related to a phenotypic trait of male sterility contains a ZIM3 KRAB repressor domain fused through a linker with the DNA binding domain (FIG. 8). The amino acid structure of the KRAB repressor domain (with the linker) is shown in FIG. 20 ((SEQ ID No. 7).

In the present embodiment of the invention, the synthetic regulator that limits the expression of a plant gene related to a phenotypic trait of male sterility contains an SRDX repressor domain of an ERF transcription factor(37) fused through the linker with the DNA binding domain. The amino acid structure of the SRDX repressor domain (with the linker) is shown in FIG. 21 (SEQ ID No. 8).

In the present embodiment of the invention, the synthetic regulator that limits the expression of a plant gene related to a phenotypic trait of male sterility contains fusion of the KRAB and SRDX domains. Four SRDX polypeptides are fused with one another through GS bridges. The amino acid structure of the KRAB::SRDX repressor domain (with the linker) is shown in FIG. 22 (SEQ ID No. 9).

### Example 5. A Male Sterility—Inducing Gene and its Silencing

Suppression of the expression of NP1 fertility genes in wheat is an example of use of the present invention. NP1 genes encode glucose-methanol-choline oxidoreductase. Their mutations (eliminated synthesis of a functional product) induce a trait of male sterility in wheat(4). Sterile wheat plants were obtained using the CRISPR system to mutate NP1 genes. In the present embodiment of the invention, a synthetic regulator is used to limit the expression of NP1 genes. When fused to the common promoter sequences, expression of all three recessive NP1 genes can be suppressed in wheat (Annex A).

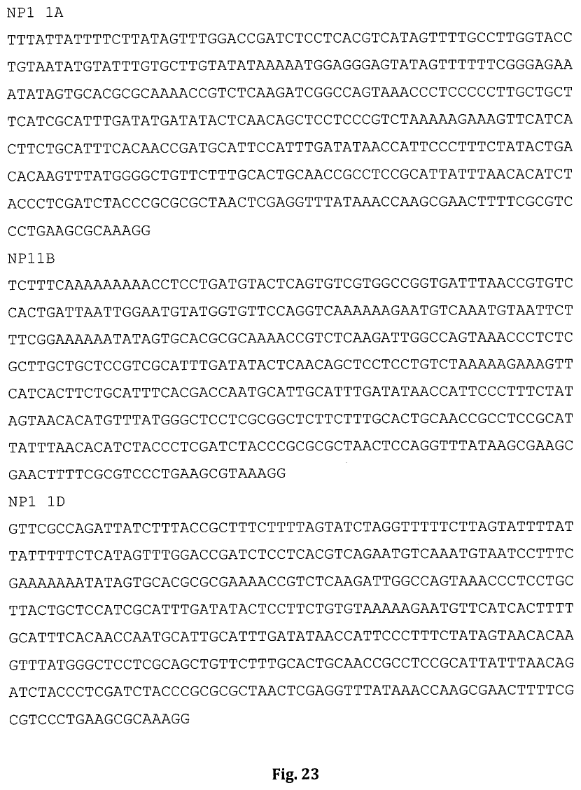

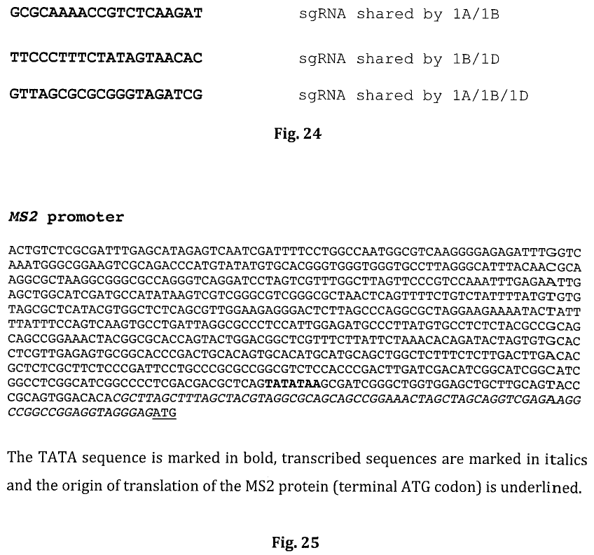

The sequences of three promoters that control the expression of NP1A, NP1B and NP1D in wheat are shown in FIG. 23 (SEQ ID No. 10, SEQ ID No. 11 and SEQ ID No. 12). The promoter sequences of NP1 genes are not identical, but they contain common elements that may be used to attach repressors (FIG. 24).

### Example 6. A Vector that Encodes Elements of the Synthetic Regulator Containing the Repressor Domain

The cassette structure of the synthetic repressor is shown in FIG. 8 and as SEQ ID No. 13. It contains a KRAB domain fused to the end of the tadCas9 coding sequence. The linker between the two repressor domains is identical as in the activator cassettes. Vectors for the synthetic repressors are obtained by replacing the VP128 activator domain in pBL-Ubi_tadCas9::VP128_sgRNA vectors. As a result, the pBL-Ubi_tadCas9::KRAB_sgRNA vector was obtained (SEQ ID No. 14). Genetic elements of sgRNA cassettes are designed and integrated similarly as discussed for activators.

### Example 7. Verification of the Efficiency of the Synthetic Regulator Containing the Activator Domain for the Activation of the MS2 Gene Promoter

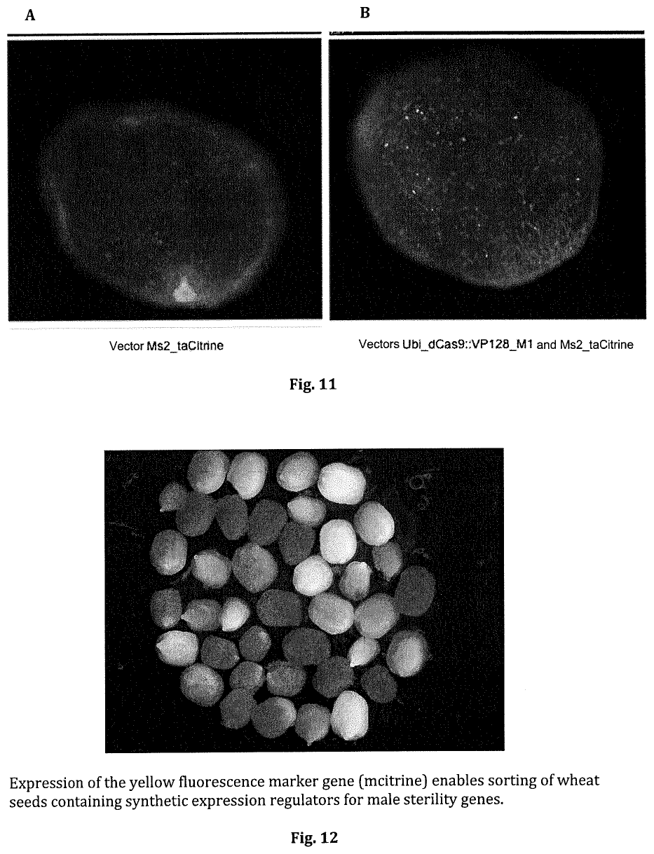

Vectors for synthetic regulators fused with a synthetic marker gene containing the MS2 promoter were used for the functional verification of an example embodiment. The vectors were incorporated into wheat cells by bombarding them with gold particles coated with the DNA of the tested vectors. The method for the preparation of gold particles for bombarding and immature wheat germs is described in Example 9. The taCitrine yellow green fluorescence gene is a visual marker of MS2 promoter activity, because its coding sequence is coupled to a MS2 promoter fragment. The MS2 promoter is not functional/active in wheat scutellum cells. Therefore, fluorescent cells are not seen on the scutellum surface after bombardment with gold particles coated with the MS2-taCitrine vector (FIG. 11, part A). However, after bombardment with gold particles coated with the MS2-taCitrine vector and the pBL-Ubi_tadCas9::VP128_sgRNA M1 vector, fluorescent spots occurred on the scutellum surface indicating activation of the taCitrine gene (FIG. 11, part B). These results confirm activation of the MS2 promoter by the tadCas9::VP 128 synthetic regulator. The procedure may be used for functional tests of synthetic regulators of the invention and variants thereof.

### Example 9. Incorporation of the Synthetic Regulator of the Invention into Plants

Methods for incorporating genetic elements into plant cells are known to plant molecular geneticists. In an example embodiment of the invention, a method for bombarding cells of immature wheat germs by DNA vectors was used. The method can be used to identify and propagate cells containing the synthetic regulator. The cells are subsequently used for the regeneration of male sterile plants.

### Cultivation of Plants

Dry seeds are sterilized in 70% ethanol for 1 minute and subsequently for 20 minutes in 1% sodium hypochlorite (Domestos:water 1:4, Unilever). After incubation in sodium hypochlorite, the seeds are washed 3 times in sterile water. The sterilized seeds are placed on Petri dishes containing filter paper soaked with water (1-1.5 mL) and incubated at 24° C. in darkness for 4 days.

Sprouted seeds are transferred into pots (19×15 cm diameter/height, 2 plants per pot) with a Substral Natural substrate (a substrate ready to use for sowing, high in humus and biocompost). The substrate is thermally processed at 80° C. for 6 h. Subsequently, 200 g (supplementing 20 liters of the substrate) of fertilizer carbonate lime containing magnesium (DOLOMIT 50 from PPHU Dolpol) is added to the substrate mixed with water. The resulting substrate is then mixed with expanded clay (from KiK Krajewscy 1972) in a 4:1 ratio. After filling the pots, 150 mL of 0.4% solution of the KRISTALON GREEN LABEL 18-18-18 fertilizer (YARA) is added to each pot.

Plants are cultivated at 20° C./15° C (day/night) with a 15/9-hour photoperiod, 40-50% humidity, illumination of 300-350 μM at the pot level, approx. 1 m from the lamp protection glass (600 W metal halide lamps and 600 W HPS lamps). The plants are regularly watered daily and fertilized once a week with 150 milliliters of 0.4% solution of the KRISTALON GREEN LABEL fertilizer. TILT-Turbo (Syngenta) is used to protect the plants against fungi. Spraying is performed as needed but not later than one week before harvesting the material for isolating immature germs.

### Preparation of Immature Embryos

Immature caryopses are harvested two weeks after flowering (when anthers emerge from the spikes) and sterilized according to the procedure (Cultivation of plants). Immature embryos are isolated with a lancet and placed on a medium containing 9% sucrose and auxin 2,4-D. Subsequently, the plant material is incubated in darkness at 23° C. for one day.

### Preparation of Gold Projectiles and Microparticle Bombardment

Gold nanoparticles (20 mg) with a size of 0.6 μm are treated several times with ethyl alcohol and subsequently suspended in sterile water. 50 μg of vector DNA, 0.1 M spermidine, and 2.5 M CaCl₂ is added to 20 mg of the nanoparticles. 400 μg of gold and 1 μg of DNA is used for one shot/bombardment. After incubation for 2 minutes at room temperature, DNA-coated nanoparticles are centrifuged at 8000 g for 15 s. After centrifugation, the gold-DNA solution is suspended in ethyl alcohol. 20 μL of the suspension of DNA-coated nanoparticles is applied on sterilized macrocarriers.

Approx. 80 immature embryos are placed in the middle of a Petri dish with a diameter of 50 mm. The Petri dish is placed 10 cm from the macrocarriers. Microparticles are delivered after creating vacuum of 27-28 mm Hg in the bombardment chamber.

The immature embryos are left in the darkness on the Petri dishes in the same medium for 1-2 days at 23° C. Subsequently, the explants are transferred onto an induction medium with lower osmotic pressure containing auxin 2,4-D and 3 mg/L phosphinothricin as a selection reagent. Embryogenic calluses are transferred into a regeneration medium containing the BAP cytokinin after 4 weeks. The Petri dishes with regenerating seedlings are incubated at 23° C. with a 16 h/8 h photoperiod (day/night). Grown regenerants are rooted in a medium containing sucrose after 3 weeks. Rooted seedlings are transferred into pots with the substrate prepared as discussed in the “Cultivation of plants” section. The plants are cultivated in the same conditions as the plants grown from seeds.

### Identification of Plants Containing the Synthetic Regulator of the Invention

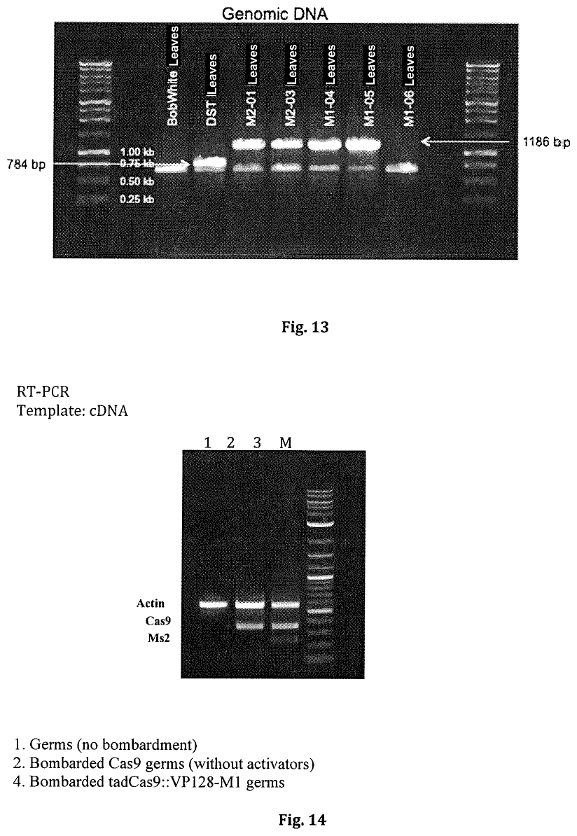

Wheat plants containing the synthetic activator were identified by analyzing genomic DNA for the content of fragments of the Ubi_tadCas9::VP128 synthetic activator gene. Therefore, genomic DNA was isolated from leaf cells of selected plants according to the DNeasy Plant Mini Kit protocol, Qiagen. To amplify the Ubi_tadCas9::VP128 gene, 20-50 ng of genomic DNA and a pair of primers specific for the amplified fragment Cas9endfw1 (CATCGAGCAGATCAGCGAGT (SEQ ID No. 29)) and U6promrw1 (GCTCTCCCCAATCG TGAACA (SEQ ID No. 30)) were used. Presence of the synthetic regulator was confirmed by amplification of a genomic DNA fragment with a length of 1186 base pairs (FIG. 13: plants M2-01, M2-03, M1-04, M1-05). The synthetic activator gene was not found in the M1-06 plant and the negative control plants—BobWhite and DST. PCR and DNA analysis on agarose gels was performed using protocols known to molecular biologists.

### Example 10. Verification of the Efficiency of the Synthetic Regulator Containing the Activator Domain for the Activation of Expression of the MS2 Male Sterility Gene

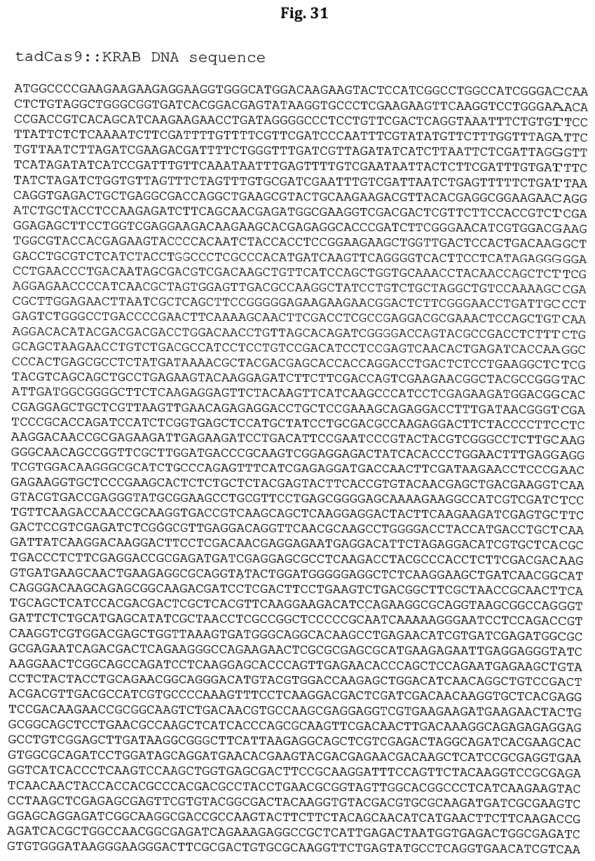

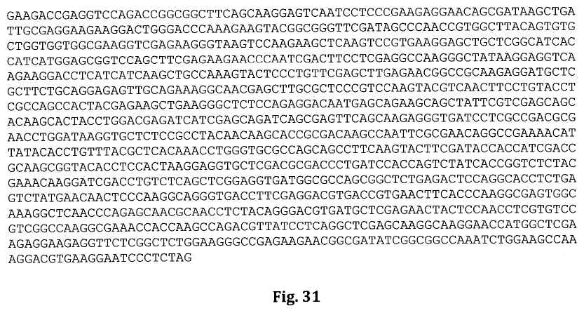

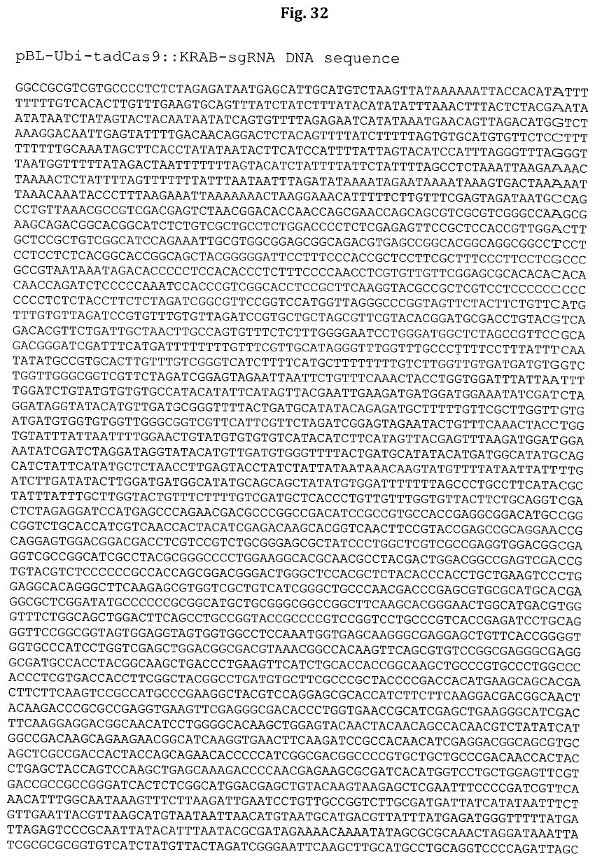

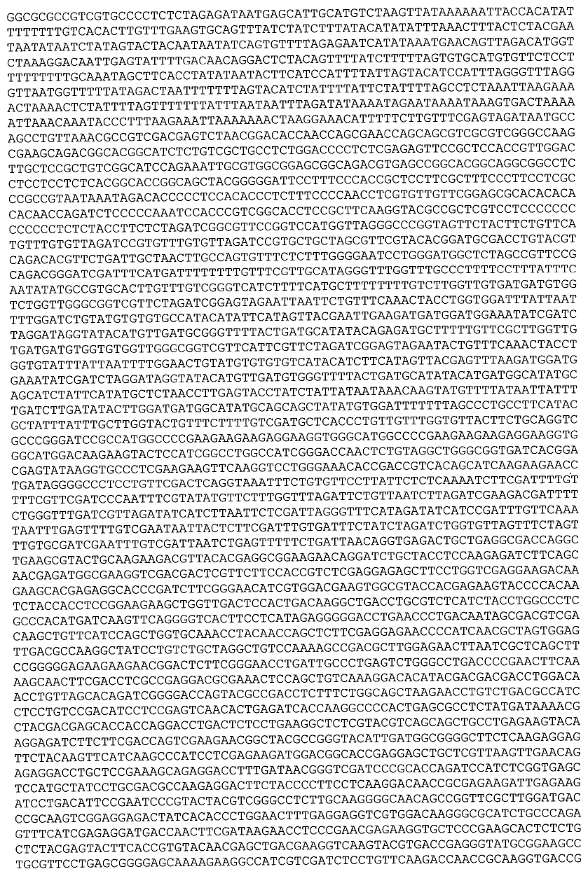

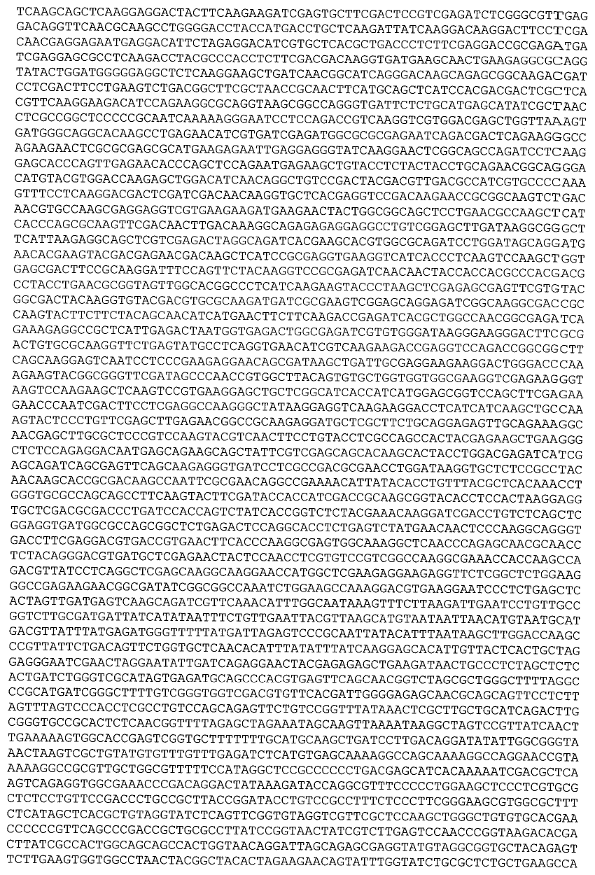

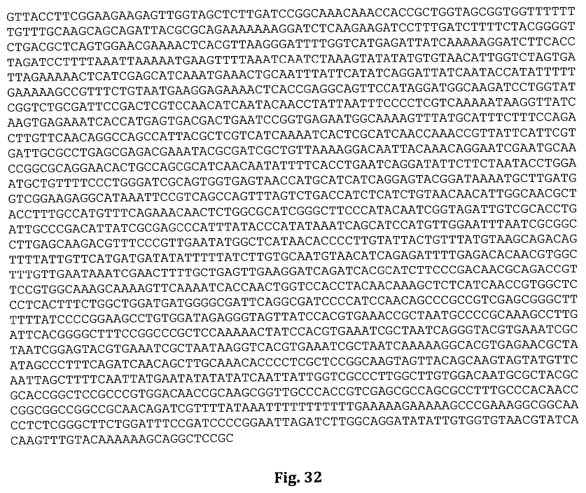

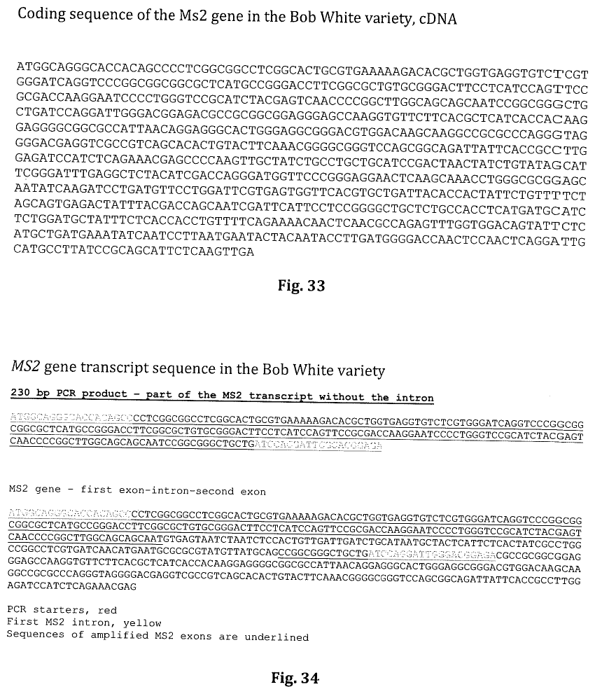

For the functional verification of an example embodiment of the invention, vectors for the synthetic regulators were used, incorporated into wheat cells using the methods described in previous examples. As shown in FIG. 14, incorporation of a synthetic activator into wheat cells causes activation of expression of the MS2 male sterility gene, manifested by the presence of mRNA transcripts of the gene (FIG. 31, SEQ ID No. 22). MS2 gene transcripts with a size of 230 base pairs emerge in wheat cells after transformation with the tadCas9::VP128-M1 activator. The transcript sequence is shown in FIG. 14 (SEQ ID No. 25). The mRNA transcript of the MS2 gene does not contain the first intron of the gene, which was removed during the mRNA maturation process (the first intron found in the MS2 gene is marked yellow). This provides direct evidence that the emerging transcripts are indeed products of the activated expression of the native MS2 gene.

### Example 11. A Process to Produce Wheat Hybrids Using the Synthetic Regulator of the Invention

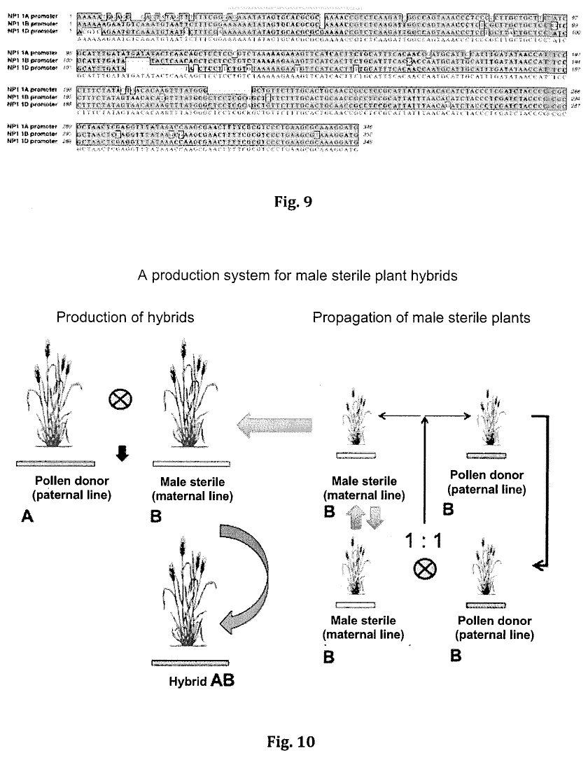

The object of the invention is a production system for hybrid plants shown in FIG. 10. The production of dioecious plant hybrids, and in particular cleistogamous plants, requires cross-breeding of two genetically distinct varieties (inbred lines) of which one is a male sterile line (maternal plant). Propagation of male sterile plants is an important element of the system. A fertile paternal plant is the pollen donor.

In the present embodiment of the invention, the sterile plants are propagated by cross-breeding with paternal plants (pollen donors), that is, plants of the same variety (A) (inbred line) (FIG. 10).

The first plant in a pair is a fertile paternal plant of a variety selected by the breeder for cross-breeding. The plant contains no extra genetic elements incorporated by genetic transformations or genome editing. The plant is a source of pollen for pollinating sterile plants, so that the sterile plants are propagated.

The second plant in a pair is a plant of the same variety having a nucleic acid sequence encoding a synthetic regulator whose expression activates the sterility gene (activator) or inactivates (repressor) endogenous fertility genes, integrated in its genome. The synthetic regulator gene is operably fused with a genetic marker that enables identification or selection of plants/seeds containing the regulatory gene. The original maternal plants are male sterile and contain the synthetic regulator. They are pollinated by the fertile paternal plants. 50% of seeds among the seeds collected from these crosses are also male sterile. In the first variant of the example embodiment of the invention, the seeds not containing the synthetic regulator may be separated by sorting the seeds in UV light. Seeds with the synthetic regulator contain a fluorescent dye marker gene (FIG. 12). Sorting of maternal plant seeds is used in other production systems for hybrids(5, 28). In the second variant of an example embodiment of the invention, fertile plants may be removed after sowing the seeds by spraying with an herbicide (an herbicide resistance marker gene fused with the synthetic regulator). In the third variant of an example embodiment of the invention, spraying with the herbicide may be performed after several production cycles in which all seeds harvested from a field are sowed without a need for the additional planting of paternal plants (plants without the synthetic regulator are pollen donors). The synthetic regulator is not transferred by pollen to the next generation, because maternal plants containing the synthetic regulator are male sterile plants. As a result of the process, homozygous plants containing the synthetic regulator do not emerge, and only heterozygous plants are propagated.

After propagation, heterozygous male sterile plants are crossbred with paternal plants of a different variety (B) (inbred line) and the AB hybrids form. All maternal plants produce seeds of the AB hybrid, and half of the seeds will produce fertile plants able to pollinate seeds in production fields. If any limitations are imposed on the cultivation of plants with synthetic regulators, the seeds may be sorted out from hybrid plant seeds thus eliminating the foreign genetic elements.

### Literature References

1. W. Akel, M. Rapp, P. Thorwarth, T. Wurschum, C. F. H. Longin, Hybrid durum wheat: heterosis of grain yield and quality traits and genetic architecture of anther extrusion. Theor. Appl. Genet. 132, 921-932 (2019).
2. X. Wan, S. Wu, X. Li, Breeding with dominant genic male-sterility genes to boost crop grain yield in the post-heterosis utilization era. Mol Plant 10.1016/j.molp.2021.02.004 (2021).
3. M. J. Milner et al., Identification of genes involved in male sterility in wheat (Triticum aestivum L.) which could be used in a genic hybrid breeding system. Plant Direct 4, e00201 (2020).
4. J. Li, Z. Wang, G. He, L. Ma, X. W. Deng, CRISPR/Cas9-mediated disruption of TaNP1 genes results in complete male sterility in bread wheat. J Genet Genomics 47, 263-272 (2020).
5. Y. Wu et al., Development of a novel recessive genetic male sterility system for hybrid seed production in maize and other cross-pollinating crops. Plant Biotechnol J 14, 1046-1054 (2016).
6. W. Cao, D. J. Somers, G. Fedak, A molecular marker closely linked to the region of Rht-D1c and Ms2 genes in common wheat (Triticum aestivum). Genome 52, 95-99 (2009).
7. F. Ni et al., Wheat Ms2 encodes for an orphan protein that confers male sterility in grass species. Nat Commun 8, 15121 (2017).
8. A. Papikian, W. Liu, J. Gallego-Bartolome, S. E. Jacobsen, Site-specific manipulation of Arabidopsis loci using CRISPR-Cas9 SunTag systems. Nat Commun 10, 729 (2019).
9. Z. Li et al., A potent Cas9-derived gene activator for plant and mammalian cells. Nat Plants 3, 930-936 (2017).
10. P. I. Thakore et al., RNA-guided transcriptional silencing in vivo with S. aureus CRISPR-Cas9 repressors. Nat Commun 9, 1674 (2018).
11. A. Piatek et al., RNA-guided transcriptional regulation in planta via synthetic dCas9-based transcription factors. Plant Biotechnol J 13, 578-589 (2015).
12. X. Wan et al., Maize Genic Male—Sterility Genes and Their Applications in Hybrid Breeding: Progress and Perspectives. Mol Plant 12, 321-342 (2019).
13. V. Djukanovic et al., Male-sterile maize plants produced by targeted mutagenesis of the cytochrome P450-like gene (MS26) using a redesigned I-Crel homing endonuclease. Plant J. 76, 888-899 (2013).
14. E. J. Tucker et al., Molecular identification of the wheat male fertility gene Ms1 and its prospects for hybrid breeding. Nat Commun 8, 869 (2017).
15. L. Reynolds et al., Repression of the HIV-1 5′ LTR promoter and inhibition of HIV-1 replication by using engineered zinc-finger transcription factors. Proc. Natl Acad. Sci. USA 100, 1615-1620 (2003).
16. R. Urrutia, KRAB-containing zinc-finger repressor proteins. Genome Biol 4, 231 (2003).
17. J. Boch et al., Breaking the code of DNA binding specificity of TAL-type III effectors. Science 326, 1509-1512 (2009).
18. K. S. Makarova et al., Evolution and classification of the CRISPR-Cas systems. Nat Rev Microbiol 9, 467-477 (2011).
19. X. Zhang et al., MiniCAFE, a CRISPR/Cas9-based compact and potent transcriptional activator, elicits gene expression in vivo. Nucleic Acids Res. 10.1093/nar/gkab 174 (2021).
20. E. Deltcheva et al., CRISPR RNA maturation by trans-encoded small RNA and host factor RNase III. Nature 471, 602-607 (2011).
21. M. Jinek et al., A programmable dual-RNA-guided DNA endonuclease in adaptive bacterial immunity. Science 337, 816-821 (2012).
22. X. Xiong, J. Liang, Z. Li, B. Q. Gong, J. F. Li, Multiplex and optimization of dCas9-TV-mediated gene activation in plants. J Integr Plant Biol 63, 634-645 (2021).
23. Y. Wang et al., Genome-wide characterization of JASMONATE-ZIM DOMAIN transcription repressors in wheat (Triticum aestivum L.). BMC Genomics 18, 152 (2017).
24. N. F. Rizvi, J. D. Weaver, E. J. Cram, C. W. Lee-Parsons, Silencing the Transcriptional Repressor, ZCT1, Illustrates the Tight Regulation of Terpenoid Indole Alkaloid Biosynthesis in Catharanthus roseus Hairy Roots. PLOS One 11, e0159712 (2016).
25. M. M. Mahfouz et al., Targeted transcriptional repression using a chimeric TALE-SRDX repressor protein. Plant Mol Biol 78, 311-321 (2012).
26. N. Alerasool, D. Segal, H. Lee, M. Taipale, An efficient KRAB domain for CRISPRi applications in human cells. Nat Methods 17, 1093-1096 (2020).
27. B. C. Stanton et al., Genomic mining of prokaryotic repressors for orthogonal logic gates. Nature chemical biology 10, 99-105 (2014).
28. X. An et al., Molecular regulation of ZmMs7 required for maize male fertility and development of a dominant male-sterility system in multiple species. Proc. Natl Acad. Sci. USA 117, 23499-23509 (2020).
29. L. S. Qi et al., Repurposing CRISPR as an RNA-guided platform for sequence-specific control of gene expression. Cell 184, 844 (2021).
30. L. S. Qi et al., Repurposing CRISPR as an RNA-guided platform for sequence-specific control of gene expression. Cell 152, 1173-1183 (2013).
31. I. Sadowski, J. Ma, S. Triezenberg, M. Ptashne, GAL4-VP16 is an unusually potent transcriptional activator.Nature 335, 563-564 (1988).
32. J. Wang et al., Overexpression of OsMYB1R1-VP64 fusion protein increases grain yield in rice by delaying flowering time. FEBS Lett 590, 3385-3396 (2016).
33. J. P. Guilinger, D. B. Thompson, D. R. Liu, Fusion of catalytically inactive Cas9 to FokI nuclease improves the specificity of genome modification. Nat. Biotechnol. 32, 577-582 (2014).
34. C. Xia et al., A TRIM insertion in the promoter of Ms2 causes male sterility in wheat. Nat Commun 8, 15407 (2017).
35. S. Gasparis et al., A simple and efficient CRISPR/Cas9 platform for induction of single and multiple, heritable mutations in barley (Hordeum vulgare L.). Plant methods 14, 111 (2018).
36. X. Liu, Y. Shangguan, J. Zhu, Y. Lu, B. Han, The rice OsLTP6 gene promoter directs anther-specific expression by a combination of positive and negative regulatory elements. Planta 238, 845-857 (2013).
37. K. Hiratsu, K. Matsui, T. Koyama, M. Ohme-Takagi, Dominant repression of target genes by chimeric repressors that include the EAR motif, a repression domain, in Arabidopsis. Plant J . 34, 733-739 (2003).

1. A synthetic expression regulator of a plant gene related to a phenotypic trait of male sterility containing a protein having a protein DNA binding domain of the plant gene related to a phenotypic trait of male sterility and a protein activator or repressor domain for the transcription process of the gene linked with the former, wherein the protein DNA binding domain forms a complex with an RNA molecule having a sequence complementary to the element that controls expression of the sterility gene.

2. An expression regulator of claim 1, characterized in that the plant gene related to a phenotypic trait of male sterility is a grain crop gene, preferably a wheat gene.

3. An expression regulator of claim 1, characterized in that the RNA molecule having a sequence complementary to the element that controls expression of the sterility gene has been selected from a group including RNA molecules known as tracrRNA and crRNA or their synthetic versions known as sgRNA being components of the CRISPR system.

4. An expression regulator of claim 1, characterized in that the protein activator domain has been selected from a group including natural domains of transcription factors, such as VP-16 and its synthetic derivatives: SunTag or TV.

5. An expression regulator of claim 1, characterized in that the protein DNA binding domain has an amino acid sequence shown as SEQ ID No. 2 (Annex 2).

6. An expression regulator of claim 1, characterized in that the protein repressor domain has been selected from a group including domains of natural repressors, such as JAZ, ZTC1, SRDX, KRAB repressor domains or prokaryotic repressor domains, such as TetR.

7. An expression regulator of claim 1, characterized in that the protein activator domain has an amino acid sequence shown as SEQ ID No. 2, SEQ ID No. 4 or SEQ ID No. 5.

8. An expression regulator of claim 1, characterized in that the RNA molecule has a nucleotide sequence shown as SEQ ID No. 3.

9. An expression cassette encoding the expression regulator defined in claims 1

10. An expression cassette of claim 1, characterized in that it has a nucleotide sequence shown as SEQ ID No. 13.

11. An expression vector containing the expression cassette of claim 9-10.

12. An expression vector of claim 11, characterized in that it has a nucleotide sequence shown as SEQ ID No. 14.

13. A process for producing male sterile plants, characterized in that an expression vector for a synthetic regulator of sterility or fertility genes is incorporated into plant cells, cells that contain the expression regulator are identified, the identified cells are propagated and subsequently used to regenerate male sterile plants, wherein preferably an expression vector defined in claims 11-12 is incorporated into the plant cells, cells that contain the expression regulator defined in claims 1-8 are identified, the identified cells are propagated and subsequently used to regenerate male sterile plants.

14. A process of claim 13, characterized in that DNA of the expression vector is incorporated into plant cells by microparticle bombardment.

15. A process of claim 13, characterized in that cells that contain the expression regulator are identified by analyzing their genomic DNA for the content of synthetic regulator gene fragments, for the presence of mRNA transcripts of the gene, preferably using PCR.

16. A process for producing a hybrid plant, characterized in that it includes cross-breeding of two genetically distinct varieties of inbred lines, wherein one of these is a male sterile line of one variety obtained using a process defined in claims 13-14, and the other one is a parental line of a different variety.

17. A process of claim 15, characterized in that the male sterile line is obtained using the synthetic regulator defined in claims 1-8 as a dominant sterility trait.
